(19) United States
(12) Patent Application Publication
(10) Pub. No.: US 2024/0058310 A1
(43) Pub. Date: Feb. 22, 2024

(54) METHODS OF TREATING DISEASES AND DISORDERS ASSOCIATED WITH CALCIUM ION SIGNALING

(71) Applicant: Temple University-Of the Commonwealth System of Higher Education, Philadelphia, PA (US)

(72) Inventors: Jonathan Soboloff, Philadelphia, PA (US); M. Raza Zaidi, Philadelphia, PA (US); Scott Gross, Philadelphia, PA (US); John Elrod, Philadelphia, PA (US)

(21) Appl. No.: 18/329,957

(22) Filed: Jun. 6, 2023

Related U.S. Application Data
(60) Provisional application No. 63/349,376, filed on Jun. 6, 2022.

(51) Int. Cl. A61K 31/426; A61P 35/04

(52) U.S. Cl. CPC A61K 31/426; A61P 35/04

(57) ABSTRACT

The invention comprises methods for treating diseases or disorders associated with reduced levels of Ca²⁺ signaling, including melanoma and other cancers.

### CROSS-REFERENCE TO RELATED APPLICATIONS

This application claims priority to U.S. Provisional Patent Application No. 63/349,376 filed on Jun. 6, 2022, the contents of which are incorporated by reference herein in their entirety.

### STATEMENT OF GOVERNMENT FUNDING

This invention was made with government support under R01GM117907, 1R01AI43256, K99DK120876, P30CA006927, and U54 CA221704(5) awarded by the National Institutes of Health. The government has certain rights in the invention.

### BACKGROUND OF THE INVENTION

Melanoma is a cancer of the cells of the melanocyte lineage, which are predominantly responsible for the melanin pigment production that defines skin and eye tone (Slominski, A., et al, 2004, Physiological Reviews, 84:1155-1228). Melanoma incidence has progressively risen over the last 30 years (Schadendorf, D., et al, 2018, The Lancet, 392:971-984), and is now the fifth most common cancer among men and women in the US (Alteri, R., 2019, Cancer Facts & FIGS. 2019). Although melanoma is easily treated by surgical resection when detected early, if undetected melanoma progresses from a radial to vertical growth phase to become an aggressive malignancy that easily metastasizes from the skin to the lymph nodes and other distal sites (Schadendorf, D., et al., 2018, The Lancet, 392:971-984). Currently, there are two major approaches to treating metastatic melanoma: targeted therapies and immunotherapies (Neves de Oliveira, B. H., et al, 2018, Medical Sciences (Basel, Switzerland), 6(1):23). Targeted therapies include inhibitors of targets in the Mitogen-Activated Protein Kinase (MAPK) pathway, including oncogenic BRAF^{V600E} and MEK1/2 (trametinib) inhibitors. These drugs transiently increase progression-free survival, although this is typically followed by drug resistance and relapse (Neves de Oliveira et al., 2018, Medical Sciences (Basel, Switzerland), 6(1):23). Additionally, though promising, MAPK pathway inhibitors inhibit immune cell function, leading to increased clinical toxicity (Boni, A., et al, 2010, Cancer Research, 70:5213). Immunotherapies were first explored as first-line melanoma treatments due to the observation that incidence of melanoma increased in immunosuppressed patients (Greene, M., et al, 1981, The Lancet, 317:1196-1199); targets of immune checkpoint inhibitors (ICIs) including Cytotoxic T-Lymphocyte Antigen 4 (CTLA4) (ipilimumab) and Programmed Cell Death-1 (PD1) (pembrolizuamab). ICI therapies in conjunction with targeted therapies are currently in use. Continued efforts to identify new strategies are needed to control this deadly disease.

Ultraviolet (UV) radiation exposure is a major risk factor in melanoma, and the rising incidence of melanoma has been linked to increased sun exposure and artificial tanning (Matthews, N. H., et al., 2017, Cutaneous Melanoma: Etiology and Therapy, Codon Publications, Chapter 1; Schadendorf, D., et al., 2018, The Lancet, 392:971-984). UV triggers melanin production in melanocytes, which protects against UV-induced damage in skin (Brenner, M. & Hearing, V. J., 2008, Photochemistry and Photobiology, 84:539-549). Conversely, UV is considered a “complete carcinogen”, affecting multiple cellular processes to promote cancer initiation and progression (D'Orazio, J., et al, 2013, International Journal of Molecular Sciences, 14:12222-12248). The most immediate consequence of UV exposure is induction of pyrimidine dimers that can cause DNA damage and/or mutations. While the evidence that UV drives melanomagenesis is undeniable, the molecular pathways linking UV and driver mutations are unclear since the most common mutations associated with melanoma, such as BRAF and NRAS mutations, do not possess the UV signature mutations. Further, while UV has long been associated with melanomagenesis, UV also has been reported to drive melanoma progression (Arisi, M., et al, 2018, Frontiers in Medicine, 5:235; Bald, T., et al, 2014, Nature, 507:109-113), marked by entry into the vertical growth phase (Guerry, D., et al, 1993, Journal of Investigative Dermatology, 100:S342-S345).

Thus, there is a need for novel therapeutics which inhibit UV-supported progression of melanomagenesis. The present invention addresses this long felt but unmet need.

### SUMMARY OF THE INVENTION

The present invention relates methods of treating a disease or disorder associated with reduced levels of Ca²⁺ signaling in need thereof, comprising administering the to the subject a composition comprising at least one modulator of store-operated Ca²⁺ entry (SOCE) in a subject in need thereof.

In some embodiments, the modulator of SOCE is a modulator of a member selected from the group consisting of Orai1, Orai2, and Orai3. In some embodiments, wherein the modulator of SOCE is a modulator of Orai1. In some embodiments, the modulator of Orai1 is 4-((5-Phenyl-4-(trifluoromethyl)thiazol-2-yl)amino)benzoic acid (IA65) or pharmaceutically acceptable salt thereof.

In some embodiments, the disease or disorder associated with reduced levels of Ca²⁺ signaling is cancer. In some embodiments, the disease or disorder associated with reduced levels of Ca²⁺ signaling is selected from the group consisting of basal cell carcinoma, squamous cell carcinoma, Merkel cell carcinoma, and melanoma.

In some embodiments, the disease or disorder is melanoma. In some embodiments, administering the composition to the subject downregulates anabolic metabolic pathways in cancerous cells. In some embodiments, administering the composition to the subject reduces invasiveness of the melanoma. In some embodiments, administering the composition to the subject reduces migration of the melanoma. In some embodiments, administering the composition to the subject reduces metastasis of the melanoma.

In some embodiments, modulator of SOCE is a modulator of a member selected from the group consisting of Orai1, Orai2, and Orai3. In some embodiments, the modulator of SOCE is a modulator of Orai1. In some embodiments, the modulator of Orai1 is 4-((5-Phenyl-4-(trifluoromethyl)thiazol-2-yl)amino)benzoic acid (IA65) or pharmaceutically acceptable salt thereof. In some embodiments, administering the composition to the subject elevates SOCE levels of cancerous cells to the levels of non-cancerous cells. In some embodiments, administering the composition to the subject downregulates anabolic metabolic pathways in cancerous cells. In some embodiments, administering the composition to the subject reduces the invasiveness of the melanoma. In some embodiments, administering the composition to the subject reduces migration of the melanoma. In some embodiments, wherein administering the composition to the subject reduces metastasis of the melanoma.

In some embodiments, administering the composition to the subject does not alter SOCE levels of non-cancerous cells.

### BRIEF DESCRIPTION OF THE DRAWINGS

The patent or application file contains at least one drawing executed in color. Copies of this patent or patent application with color drawing(s) will be provided by the Office upon request and payment of the necessary fee.

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1, comprising FIG. 1A through FIG. 1H, representative experimental results demonstrating that UV exposure suppresses SOCE and enhances invasion. FIG. 1A depicts representative traces showing the effect of UV on SOCE in 8 human melanoma cell lines. Cells were incubated overnight before loading with Fura2-AM; cells were treated with 2 μM Tg for 15 minutes in the absence of extracellular Ca²⁺ (not shown) before the addition of 1 mM Ca²⁺. Dashed lines indicate SEM. FIG. 1B depicts representative UV-dependent differences in SOCE determined by comparing the maximal change in Fura2 ratio between UV-treated and untreated cells after the addition of extracellular Ca²⁺. Biological replicates are represented by dots, with each replicate based upon data obtained from 10 to 60 individual cells. Data were analyzed by two-way ANOVA with multiple comparisons (UV effect, p<0.0001; effect of cell line, p>0.05; interaction, p>0.05). FIG. 1C depicts representative long-term analysis of UACC257 and WM983 cells incubated for 1 hour with Rhod2-AM at 37 C and then treated as described for FIG. 1A, except the incubation periods were extended as depicted (N≥3). Data were analyzed by two-way ANOVA with multiple comparisons (UV effect, p<0.01; effect of cell line, p<0.05; interaction, p>0.05). FIG. 1D depicts representative images and quantification of UV-treated cells placed in Matrigel® transwell inserts in OptiMEM reduced serum media; DMEM with 10% FBS was used as a chemoattractant. Crystal violet-stained invaded cells were measured 20 hours after initiation of assay (N≥4). Sample images for each condition are shown above the corresponding bar. Data were analyzed by two-way ANOVA with multiple comparisons (UV effect, p<0.0001; Effect of cell line, p>0.05; interaction, p<0.01). FIG. 1E depicts representative SOCE measured in B16N murine melanoma cells as in FIG. 1A. FIG. 1F depicts SOCE results from FIG. 1E which were quantified as in FIG. 1B (N≥15). Data were analyzed by one-way ANOVA with multiple comparisons (p<0.05). FIG. 1G depicts representative invasiveness of B16N cells as determined in FIG. 1F, (N≥9). Data were analyzed by one-way ANOVA with multiple comparisons (p=0.0001). FIG. 1H depicts representative imaging of abdominal organs, peritoneal wall, and crude abdomen after 2 weeks of tumor cell in vivo growth after B16N cells that were exposed to UV and incubated for the indicated time before subcutaneous injection into the right flank of syngeneic C57Bl6 mice (N≥9). *, p<0.05; **, p<0.01; ***, p<0.001; ****, p<0.0001.

FIG. 2, comprising FIG. 2A through FIG. 2D, depicts representative calcium data from UVT-treated human melanoma cells from FIG. 1. FIGS. 2A and 2B depict representative examples of Ca²⁺ measurements began in the presence of Ca²⁺ (1 mM). Ca²⁺ was removed at least 1 minute prior to the addition of Tg (2 μM) followed by the re-addition of Ca²⁺ (1 mM). FIG. 2C depicts representative basal Ca²⁺, the average Fura2 ratio during the first minute. FIG. 2D depicts representative Tg-induced Ca²⁺ release, quantified by calculating the “area under the curve” to determine total Ca²⁺ release after the addition of Tg, but prior to the re-addition of Ca²⁺. Data (n≥3) were analyzed by one-way ANOVA. *, p<0.05; **, p<0.01; ***, p<0.001; ****, p<0.0001.

FIG. 3 depicts representative effects of UV and SOCE suppression on proliferation. WM983, UACC258, and B16N cells were exposed to 175 J/m² UVR followed by 1- to 5-week incubations in growth optimal media, respectively. O1C78 (WM983) and O1C44 (B16N) are melanoma cells stably expressing Orai1-WT-CFP. To measure proliferation, equal numbers of cells were plated in 96-well plates and incubated for 3 days before being treated with 10% WST-1 for 1 hour prior to absorbance reading. Data were analyzed by one-way ANOVA. ***, p<0.001.

FIG. 4, comprising FIG. 4A through FIG. 4M, depicts representative data demonstrating that partial SOCE suppression increases invasiveness. FIG. 4A depicts representative traces of SOCE in Fura-2 loaded B16N cells treated with the indicated concentrations of BTP2 for 15 minutes, in the presence of Tg. A representative trace of UV5 B16N is also shown for reference. FIG. 4B depicts quantification of SOCE BTP2 dose response from FIG. 4A, by non-linear regression (n=6). FIG. 4C depicts imaging and quantification of BTP2-treated B16N cells plated on Matrigel® transwell inserts in OptiMEM reduced serum media; DMEM with 10% FBS was used as a chemoattractant. Crystal violet-stained invaded cells were measured 20 hours after initiation of assay (N≥3). Sample images for each condition are shown above the corresponding bar. Data were analyzed by one-way ANOVA with multiple comparisons (p<0.01). FIG. 4D depicts quantification of cells which were exposed to UV for 2 hours and then treated with indicated concentrations of BTP2 as described FIGS. 4A and 4B, followed by plating on Matrigel® transwell inserts as described in FIG. 4C. FIG. 4E depicts quantification of cells which were exposed to UV for 5 hours as described in FIG. 4D. FIG. 4F depicts representative traces of SOCE in 2 Orai1-CFP-expressing cell lines exhibiting SOCE suppression similar to BTP2 (40 nM). FIG. 4G depicts relative SOCE suppression observed in Orai1-expressing or UV5 B16N cells (N≥5). Data were analyzed by one-way ANOVA with multiple comparisons (p<0.01). FIG. 4H depicts representative transwell migration assays of Parental, UV2, UV5, O1C3 and O1C44 B16N cells as described in FIG. 2C (N≥6). Data were analyzed by one-way ANOVA with multiple comparisons (p<0.001). FIG. 4I depicts representative traces of SOCE in B16N cells stably expressing the dominant negative form of Orai1-CFP (E106A; O1-DN). FIG. 4J depicts representative transwell migration assays of B16N parental versus O1-DN-expressing cells as described in (C) (N≥4). Data were analyzed by unpaired T test (p<0.05). FIG. 4K depicts representative quantitation cell-free area of B16N cell monolayers 1, 10, and 20 hours after being scratched. where, control, UV2, UV5, and O1C44 were incubated overnight in the presence or absence of 2 μM IA65. Data were analyzed by two-way ANOVA. In the absence of IA65, SOCE suppression effect was p<0.05; effect of time was p<0.0001; interaction was p>0.05. In the presence of IA65, SOCE suppression effect was p>0.05; effect of time was p<0.0001; interaction was p<0.01. FIG. 23 depicts representative imaging of B16N parental and O1C44 cells after being subcutaneously injected in the right flank of syngeneic C57BI6 mice, revealing the presence of O1C44 (but not B16N-WT) cells in abdominal organs and within the peritoneal wall. FIG. 4M depicts representative H&E stains of the peritoneal wall, revealing the presence of melanoma cells in the peritoneal wall. *, p<0.05; **, p<0.01; ***, p<0.001; ****, p<0.0001.

FIG. 5, comprising FIG. 5A through FIG. 5D, depicts representative SOCE suppression in human melanoma cells and an apparent “Goldilocks effect”. FIG. 5A depicts representative traces of SOCE in Fura-2 loaded WM983 cells treated with the indicated concentrations of BTP2 for 15 minutes in the presence of Tg. Data are representative of 5 experiments. UV2 is shown for comparison. FIG. 5B depicts representative results of invasion assays in BTP2-treated WM983 cells that were plated on Matrigel® transwell inserts in OptiMEM reduced serum media; DMEM with 10% FBS was used as a chemoattractant. Invaded cells were measured stained with crystal violet after 20 hours (N≥3). UV2 and UV5 WM983 cells are also shown for comparison. FIG. 5C depicts representative traces of SOCE in parental and an Orai1-CFP-expressing WM983 (O1C78) exhibiting SOCE suppression similar to BTP2 (80 nM). FIG. 5D depicts representative results of invasion assays, performed as in FIG. 5B, comparing the number of invaded cells in O1C78 cells to either untreated parental WM983 cells or WM983 cells exposed to UV irradiation and allowed to recover for 2 weeks (N≥4). Data were analyzed by one-way ANOVA. *, p<0.05; **, p<0.01; ***, p<0.001; ****, p<0.0001.

FIG. 6 depicts representative images of cell migration plotted in FIG. 4K. Untreated, UV5 or Orai1 overexpressing clones O1C44 and O1C3 B16N cells were plated in a monolayer overnight followed by scratching with a p10 pipette to create a cell-free zone. Migration into this zone was monitored by time lapse bright field microscopy for 20 hours at 37° C. and 5% CO₂. Representative images at times 0, 10, and 20 hours are depicted, with cell-free areas demarcated by yellow lines.

FIG. 7, comprising FIG. 7A and FIG. 7B, depicts representative toxicity of IA65 on melanoma cells. FIG. 7A depicts representative viability data of control WM983, UV2, and UV5 cells incubated in optimal growth media after which equal numbers of cells were plated in 24-well plates and incubated overnight before being treated with 0.4% Trypan Blue and comparing the Number of stained vs unstained cells. FIG. 7A depicts representative viability data of control B16N, UV2, and UV5 cells incubated in optimal growth media after which equal numbers of cells were plated in 24-well plates and incubated overnight before being treated with 0.4% Trypan Blue and comparing the number of stained vs unstained cells. Data were analyzed by one away ANOVA.

FIG. 8, comprising FIG. 8A through FIG. 8F, depicts representative suppression of UV-induced invasiveness by enhanced SOCE. FIG. 8A depicts representative traces of SOCE in Fura2-loaded cells treated with 2 μM IA65 or vehicle (Control) in the presence of Tg in nominally Ca²⁺-free media for 15 minutes prior to the addition of Ca²⁺ (1 mM). FIG. 8B depicts quantitation of SOCE from FIG. 8A (N≥4); data were analyzed by two-way ANOVA (effect of UV, p>0.05; IA65, p<0.001; interaction was p<0.01). FIG. 8C depicts representative control, UV2 and UV5 cells that were treated with IA65 (0 or 2 μM) for 15 minutes before being placed in Matrigel® transwell inserts in OptiMEM reduced serum media; DMEM with 10% FBS was used as a chemoattractant. Crystal violet-stained invaded cells were measured 20 hours after initiation of assay (N≥4). Sample images for each condition are shown above the corresponding bar. Data were analyzed by two-way ANOVA (effect of UV, p<0.01; IA65, p<0.0001; interaction was p<0.01). FIG. 8D depicts representative images of scratched confluent layers of WM983 cells treated as indicated followed by a 0-, 10-, or 20-hour incubation at 37° C. with 5% CO₂ in the absence (−) or presence (+) of 2 μM IA65. FIG. 8E depicts quantification of cells in FIG. 8D in the absence IA65, where SOCE suppression effect was p<0.05; effect of time was p<0.0001; interaction was p<0.05. FIG. 8F depicts quantification of cells in FIG. 8D in the presence of IA65, where SOCE suppression effect was p>0.05; effect of time was p<0.0001; interaction was p>0.05. *, p<0.05; **, p<0.01; ***, p<0.001; ****, p<0.0001.

FIG. 9, comprising FIG. 9A through FIG. 9F, depicts representative reversal of SOCE suppression by IA65 and the inhibition of invasiveness in B16N melanoma cells. FIG. 9A depicts representative traces of SOCE in Fura2-loaded cells that had been plated on glass coverslips overnight before exposure to 175 J/m2 UVR and incubated under growth-optimal conditions for 0 (control), 2 (UV2), or 5 (UV5) weeks, and treated with IA65 (0 or 2 μM) in the presence of Tg in nominally Ca²⁺-free media for 15 minutes prior to the addition of Ca²⁺ (1 mM). FIG. 9B depicts quantitation of SOCE from FIG. 9A. FIG. 9C depicts representative migration data from control, UV2, and UV5 cells that were treated with IA65 (0 or 2 μM) for 15 minutes before being placed in Matrigel® transwell inserts; the number of cells migrating through Matrigel® was quantified as described in FIG. 1F (N≥3). FIG. 9D depicts representative traces of SOCE in Fura2-loaded cells treated with IA65 (0 or 2 μM) in the presence of Tg in nominally Ca²⁺-free media for 15 minutes prior to the addition of Ca²⁺ (1 mM). FIG. 9E depicts quantitation of SOCE from FIG. 9D (N≥4). FIG. 9F depicts representative migration data from parental and O1C44 cells treated with IA65 (0 or 2 μM) for 15 minutes before being placed in Matrigel® transwell inserts; the number of cells migrating through Matrigel® was quantified as described in FIG. 1F (N≥6).

FIG. 10, comprising FIG. 10A through FIG. 10F, representative Orai and STIM family gene expression patterns in response to SOCE suppression. FIG. 10A depicts representative Orai expression in control WM983, UV2, UV5, and O1C78 cells. FIG. 10B depicts representative Orai expression in control UACC27, UV2, and UV5 cells. FIG. 10C depicts representative Orai expression in control B16N, UV2, UV5, and O1C44 cells. FIG. 10D depicts representative STIM expression in control WM983, UV2, UV5, and O1C78 cells. FIG. 10E depicts STIM expression in control UACC27, UV2, and UV5 cells. FIG. 10F depicts representative STIM expression in control B16N, UV2, UV5, and O1C44 cells. UV2 and UV5 cells are melanoma cells that were exposed to 175 J/m² UVR followed by 2- or 5-week incubations in growth optimal media, respectively. O1C78 (WM983) and O1C44 (B16N) are melanoma cells stably expressing Orai1-WT-CFP. 18S rRNA was used as a housekeeping gene; data (N≥4) are presented as normalized to control. UV data were analyzed by one-way ANOVA; Orai1 overexpressing clones were compared to control by t-test.

FIG. 11, comprising FIG. 11A through FIG. 11K, depicts representative correlation of mevalonate pathway upregulation with UV-induced SOCE suppression. FIG. 11A depicts representative traces of SOCE in WM983 cells that were plated on glass coverslips overnight before exposure to 175 J/m² UV and then incubated under growth optimal conditions for 0 (control) or 2 (UV) weeks followed by plating on glass coverslips, Fura2 loading and administration of Tg in nominally Ca²⁺-free media for 15 minutes prior to the addition of Ca²⁺ (1 mM), as in FIG. 1A. FIG. 11B depicts representative traces of SOCE in UACC257 cells as described in FIG. 11A. FIG. 11C depicts representative long-term analysis of UACC257 and WM983 cells incubated for 1 hour with Rhod2-AM at 37° C. and then treated as described for FIGS. 11A and 4B, except the incubation periods were extended as depicted. FIG. 11D depicts representative principal component (PC) analysis showing the variability between the replicates. FIG. 11E depicts a representative heatmap comparing genes significantly differing in WM983 and UACC257 cells two weeks after UV exposure (p<0.01, q<0.01). FIG. 11F depicts a representative Venn diagram showing the genes significantly changed due to UV in WM983 but not significantly changed in UACC257 cells. FIG. 11G depicts representative percentages of tumors in which the mevalonate pathway is increased by tumor type; data were obtained from TCGA. The numbers indicate the total number of samples available for each tumor type. FIG. 11H depicts representative imaging of WM983 and UACC257 human melanoma cells were transfected with Perfringolysin O (PFO)-mCherry D4H cholesterol sensor and plated on glass coverslips. Cells were exposed to UV (0 or 175 J/m²) and incubated for 0, 2, or 5 weeks as indicated or exhibited Orai1-WT-CFP as described in FIG. 2F. FIG. 11I depicts quantification of fluorescence intensity from cells analyzed in FIG. 11H measured and quantified using LASX analysis software (N≥8). FIG. 11J depicts representative imaging of WM983 and O1C78 human melanoma cells transfected with Perfringolysin O (PFO)-mCherry D4H cholesterol sensor as in FIG. 11H. FIG. 11K depicts quantification of fluorescence intensity of FIG. 11J as in FIG. 11I. Data in FIG. 11I were analyzed using one-way ANOVA with multiple comparisons (WM983, p<0.001; UACC257 p>0.05). *, p<0.05; **, p<0.01; ***, p<0.001; ****, p<0.0001.

FIG. 12, comprising FIG. 12A through FIG. 12E, depicts representative data demonstrating that cholesterol production is a key mediator of UV-induced metastasis. WM983 cells were plated on glass coverslips overnight before exposure to 175 J/m² UV and then incubated under growth optimal conditions for 0 (control), 2 (UV2), or 5 (UV5) weeks. FIG. 12A depicts representative traces of SOCE following UV exposure and incubation, treatment with or without the cholesterol synthesis inhibitors TAK475 or compactin overnight followed by Fura2 loading and administration of Tg in nominally Ca²⁺-free media for 15 minutes prior to the addition of Ca²⁺ (1 mM). FIG. 12B depicts SOCE quantification from FIG. 12A. Comparison was by two two-way ANOVA (N≥5; cholesterol inhibition, p=0.0014; incubation time, p>0.05; interaction, p<0.0001). FIG. 12C depicts representative images and quantification of UV5 or control WM983 cells placed in Matrigel® transwell inserts in OptiMEM reduced serum media with or without TAK475 or compactin; DMEM with 10% FBS was used as a chemoattractant. Crystal violet-stained invaded cells were measured 20 hours after initiation of assay (N≥4). Data were analyzed by two-way ANOVA (cholesterol inhibition, p=0.0004; incubation time, p>0.05; interaction, p=0.0002). FIG. 12D depicts representative quantification of invasion area of B16N murine melanoma cells treated with or without UV and incubated for 5 weeks. Ldlr^{-/-} C57Bl6 mice were fed with regular chow or high fat diet for 2 weeks prior to administration of syngeneic B16N cells (N=10). The area of cell invasion into and through peritoneal wall measured using Aperio Imagescope software and analyzed by two-way ANOVA (high fat diet, p>0.05; UV exposure, p>0.05; interaction, p<0.05). FIG. 12E depicts representative images of invasion into the peritoneal wall. *, p<0.05; **, p<0.01; ***, p<0.001; ****, p<0.0001.

FIG. 13, comprising FIG. 13A and FIG. 13B, depicts representative data demonstrating a lack of efficacy by cholesterol inhibitors on SOCE in UACC257 cells. FIG. 13A depicts representative traces of SOCE in UACC257 cells that were plated on glass coverslips overnight, exposed to 175 J/m² UV, grown under optimal growth conditions for control or 5 weeks (UV5), plated on glass coverslips and treated with or without the cholesterol synthesis inhibitors TAK475 or compactin overnight, loaded with Fura2, incubated with Tg in nominally Ca²⁺-free media for 15 minutes, then exposed to Ca²⁺ (1 mM). FIG. 13B depicts quantitation of FIG. 13A. Comparison was by two-way ANOVA (N≥5; CHL inhibition was p=0.0014; incubation time, p>0.05; interaction was p<0.0001).

FIG. 14, comprising FIG. 14A through FIG. 14H, depicts RNAseq data demonstrating that SOCE suppression drives invasion. FIG. 14A depicts a representative heatmap of gene expression differences from RNA extracted from UV2, UV5, O1C44, or control B16N murine melanoma cells (3 biological replicates per condition). FIG. 14B depicts a representative Venn diagram comparing genes significantly changed in B16N cells 2 weeks (UV2) or 5 weeks (UV5) after exposure to those change by Orai1 overexpression (O1C44) (p<0.01). FIG. 14C depicts representative glucose uptake measured by luciferase-based assay (N≥12). Data were analyzed by one-way ANOVA (B16N, p<0.0001; WM983, p<0.0001; UACC257, p>0.05). FIG. 14D depicts representative oxygen consumption rate (OCR) of control, UV5, and O1C44 B16N melanoma cells (N≥15) control, UV5, and O1C78 B16N melanoma cells (N≥10), and control, UV5, and O1C44 B16N melanoma cells (N≥12) using an Agilent Seahorse XF96. Data were analyzed by one-way ANOVA (p<0.0001). FIG. 14E depicts representative extracellular acidification rate (ECAR) of the cells discussed in FIG. 14D. FIG. 14F depicts representative basal respiration and glycolysis of the cells discussed in FIG. 14D. FIG. 14G depicts representative qPCR data for 1E6 WM983 or WM983 cells that were treated with 50 μM siRNA targeting human OGT (Origene). FIG. 14H depicts representative imaging and quantification of B16N or WM983 cells that were placed in Matrigel® transwell inserts in OptiMEM reduced-serum media, with or without OSMI1 (40 mM); DMEM with 10% FBS was used as a chemoattractant. Crystal violet-stained invaded cells were measured 20 hours after initiation of assay (N≥4). Data were analyzed by two-way ANOVA (OSMI1 effect was p<0.0001 in B16N and WM983 cells). *, p<0.05; **, p<0.01; ***, p<0.001; ****, p<0.0001.

FIG. 15, comprising FIG. 15A through FIG. 15D, depicts representative RNAseq analysis depicted in FIG. 14. FIG. 15A depicts representative PCA analysis of Orai1 overexpressing cells. FIG. 15B depicts representative PCA analysis of UV2 B16N cells. FIG. 15C depicts representative PCA analysis of UV5 B16N cells. FIG. 15D depicts a representative heatmap comparing the genes altered in O1C44 and UV5 cells relative to B16N WT cells.

FIG. 16, comprising FIG. 16A through FIG. 16F, depicts representative analysis of patient surgical explants, revealing the clinical relevance of low SOCE and cholesterol. Cells were isolated from tumors using 50-micron Medicons and the Medimachine system and grown for 3 days before experimentation to eliminate non-melanoma cells. FIG. 16A depicts representative examples of SOCE responses in patient samples following the addition of Ca²⁺ (1 mM). Plated control or UV-irradiated (175 J/m²) cells were incubated overnight on glass coverslips prior to Fura2 loading and administration of Tg in Ca²⁺-free media for 15 minutes. FIG. 16B depicts representative SOCE in different patient samples of FIG. 16A. The effect of UV on SOCE was determined by one-way ANOVA (p>0.05). FIG. 16C depicts a representative comparison of SOCE in cells collected from both primary tumors and metastatic lymph nodes from the same patient (from the patients analyzed in FIGS. 7A and 7B). FIG. 16D depicts representative SOCE in samples exhibiting NRAS (8 of 25 samples), BRAF (5 of 25 samples), TERT promoter (6 of 25 samples), NF1 (5 of 25 samples), and CDKN2A (6 of 25 samples) alterations compared to samples that expressed WT or the unaltered gene. FIG. 16E depicts quantification of the change in SOCE of single cells from patient samples which were treated with or without the indicated drug in the presence of Tg in nominally Ca²⁺-free media for 15 minutes prior to the addition of Ca²⁺ (1 mM). Each symbol within the bar represents a different tested patient specimen. Data were analyzed by the paired T test (Tak475, p=0.046; compactin, p=0.0032; IA65, p=0.212). FIG. 16F depicts representative invasion of patient cells that were placed in Matrigel® transwell inserts in OptiMEM reduced serum media with or without TAK475 (10 μM), compactin (2 μM), IA65 (2 μM) or OSMI1 (40 μM); DMEM with 10% FBS was used as a chemoattractant. Crystal violet-stained invaded cells were measured 20 hours after initiation of assay. Each dot represents 1 patient sample. Data analyzed by two-way ANOVA (cholesterol inhibition was p<0.0001; patient samples were p<0.001; interaction was p<0.01). For IA65, (inhibition was p<0.0001; patient samples were p<0.01; interaction was p<0.0001). For OSMI1, (inhibition was p<0.05; patient samples were p>0.05; interaction was p<0.05). *, p<0.05; **, p<0.01; ***, p<0.001; ****, p<0.0001.

### DETAILED DESCRIPTION

In one aspect, the present invention is directed to methods for the treatment of diseases and disorders related to reduced Ca²⁺ signaling, for example basal cell carcinoma, squamous cell carcinoma, Merkel cell carcinoma, and melanoma. In some embodiments, the modulation of SOCE is accomplished through the administration of a composition comprising at least one modulator of SOCE. In some embodiments, the modulation of SOCE is accomplished through the administration of a modulator of a member selected from the group consisting of Orai1, Orai2, and Orai3. In some embodiments, the modulator of SOCE is a modulator of Orai1, for example, 4-((5-Phenyl-4-(trifluoromethyl)thiazol-2-yl)amino)benzoic acid (IA65).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The phrase “activate,” as used herein, means to increase a molecule, a reaction, an interaction, a gene, an mRNA, and/or a protein's expression, stability, function, or activity by a measurable amount. Activators are compounds that, e.g., bind to, increase stimulation, activation, activate, sensitize, or upregulate a protein, a gene, and an mRNA stability, expression, function and activity, e.g., agonists.

The term “agent” includes any substance, metabolite, molecule, element, compound, entity, or a combination thereof. It includes, but is not limited to, e.g., protein, oligopeptide, small organic molecule, polysaccharide, polynucleotide, and the like. It can be a natural product, a synthetic compound, a chemical compound, or a combination of two or more substances. Unless otherwise specified, the terms “agent,” “substance,” and “compound” can be used interchangeably. Further, a “test agent” or “candidate agent” is generally a subject agent for use in an assay of the invention.

In the context of the present disclosure, an “agonist” is defined as a compound that increases the basal activity of a receptor (i.e. signal transduction mediated by the receptor). The term “agonist” as used herein refers to a drug which binds to a receptor and activates it, producing a pharmacological response (contraction, relaxation, secretion, enzyme activation, etc.).

An “antagonist” is defined as a compound, which blocks the action of an agonist on a receptor. A “partial agonist” is defined as an agonist that displays limited, or less than complete, activity such that it fails to activate a receptor in vitro, functioning as an antagonist in vivo. The term “antagonist” as used herein refers to a drug which attenuates the effect of an agonist. It may be competitive (or surmountable), i.e., it binds reversibly to a region of the receptor in common with an agonist, but occupies the site without activating the effector mechanism. The effects of a competitive antagonist may be overcome by increasing the concentration of agonist, thereby shifting the equilibrium and increasing the proportion of receptors which the agonist occupies. However, it is now known that certain antagonists can affect receptor trafficking and therefore improve agonist actions indirectly.

The term “assessing” includes any form of measurement, and includes determining if an element is present or not. The terms “determining,” “measuring,” “evaluating,” “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent). “Assessing activity” includes determining the amount of activity, and/or determining whether an activity has occurred (i.e., whether an activity is present or absent). The term “binding” refers to a direct association between at least two molecules, due to, for example, covalent, electrostatic, hydrophobic, ionic and/or hydrogen-bond interactions.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “downstream” or “upstream” with respect to a signaling pathway is based on epistatic relationships in a linear signaling cascade: if “A” activates “B” and “B” activates “C”, the “A” is upstream of “B” and “B” is upstream of “C”. Similarly, “B” is downstream of “A” and “C” is downstream of “B”.

The term “expression” as used herein is defined as the transcription and/or translation of a particular nucleotide sequence.

The term “interact” or “interaction” refers to a measurable chemical or physical interaction between two components, such as a target molecule and a candidate molecule, that is capable of affecting the structure and/or composition of at least one of the components, such as a target molecule, a candidate molecule or both such that the biological activity of at least one of the components, such as the target molecule, the candidate molecule or both, is affected. Interactions capable of affecting the structure and/or composition of a component include, but are not limited to, reactions resulting in the formation of one or more covalent bonds, resulting in the breaking of one or more covalent bonds, electrostatic associations and repulsions, formation and/or disruption of hydrogen bonds, formation and/or disruption of electrostatic forces such as dipole-dipole interactions, formation and/or disruption of van der Waals interactions or processes comprising combinations of these.

The term “invade”, “invasion”, or “invasiveness” refers to a cancer or cancerous cells spreading beyond the original tissue in which it/they developed, particularly the blood/lymphatic vessels and colonization of distal organs, and measured by means including, but not limited to, Boyden chamber assay, transwell invasion assay, and monitoring of implanted cancer or cancerous cells implanted in healthy tissue.

An “inverse agonist” is defined as a compound that decreases the basal activity of a receptor.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

The term “metastasis” or “metastatic” in the context of the present disclosure refer to the spread of cancer from the primary site of origin into different areas of the body.

The term “migration” in the context of the present disclosure is defined as a process by which cells move from one place to another, and can be measured by means such as a wound assay. Migration is an integral component of the processes of invasion and metastasis.

In the context of the present disclosure, a “modulator” is defined as a compound that is an agonist, a partial agonist, an inverse agonist or an antagonist of a cellular process, GPCR, NHR, microorganism, microbial metabolite thereof, protein function, or any combination thereof. A modulator may increase the activity of the cellular process, GPCR, NHR, microorganism, microbial metabolite thereof, protein function, or any combination thereof, or may decrease the activity of the cellular process, GPCR, NHR, microorganism, microbial metabolite thereof, protein, function thereof, or any combination thereof.

“Molecule” refers to a collection of chemically bound atoms with a characteristic composition. As used herein, a molecule can be neutral or can be electrically charged. The term molecule includes biomolecules, which are molecules that are produced by an organism or are important to a living organism, including, but not limited to, proteins, peptides, lipids, DNA molecules, RNA molecules, oligonucleotides, carbohydrates, polysaccharides, glycoproteins, lipoproteins, sugars and derivatives, variants and complexes of these, including labeled analogs of these having one or more vibrational tag. The term molecule also includes candidate molecules, which comprise any molecule that it is useful, beneficial or desirable to probe its capable to interact with a molecule such as a target molecule. Candidate molecules include therapeutic candidate molecules which are molecules that may have some effect on a biological process or series of biological processes when administered. Therapeutic candidate molecules include, but are not limited to, drugs, pharmaceuticals, metabolites, potential drug candidates and metabolites of drugs, biological therapeutics, potential biological therapeutic candidates and metabolites of biological therapeutics, organic, inorganic and/or hybrid organic-inorganic molecules that interact with one or more biomolecules, molecules that inhibit, decrease or increase the bioactivity of a biomolecule, inhibitors, ligands and derivatives, variants and complexes of these. The term molecule also includes target molecules, which comprise any molecule that it is useful, beneficial or desirable to probe its capable to interact with a molecule such as a candidate molecule. Target molecules useful for identifying, characterizing and/or optimizing therapeutics and therapeutic candidates comprise biomolecules, and derivatives, variants and complexes of biomolecules. The term molecule also includes competitive binding reference molecules. Competitive binding reference molecules useful in the present invention are molecules that are known to bind, at least to some extent, to a target molecule, and in some embodiments comprise a known drug, biological therapeutic, biomolecule, lead compound in a drug discovery program, and derivatives, variants, metabolites and complexes of these.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “pharmaceutically acceptable” in the context of the present disclosure refers to a substance that is acceptable for use in pharmaceutical applications from a toxicological perspective and does not adversely interact with the active ingredient.

The term “promoter” as used herein is defined as a DNA sequence recognized by the synthetic machinery of the cell, or introduced synthetic machinery, required to initiate the specific transcription of a polynucleotide sequence. As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, express the gene product in a temporally specific manner, a spatially specific manner or be constitutive.

“Screening” referred to in the present invention includes not only so-called first screening for identifying a compound of the present invention among a plurality of candidate compounds, but also a counter screen for identifying a compound of the present invention among a plurality of candidate compounds.

A “therapeutic” treatment is a treatment administered to a subject who exhibits signs or symptoms of pathology, for the purpose of diminishing or eliminating those signs or symptoms.

As used herein, “treating a disease or disorder” means reducing the frequency with which a sign or symptom of the disease or disorder is experienced by a patient.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based in part on the discovery that restoration of store-operated Ca²⁺ entry (SOCE) reduces the progression of cancer. Accordingly, in some embodiments the invention is directed towards reduction in the invasion of melanoma. In other embodiments the invention is directed towards reduction in the migration of melanoma. Other embodiments of the invention are directed towards reduction in the metastasis of melanoma.

In certain embodiments, the invention is directed towards the use of 4-((5-Phenyl-4-(trifluoromethyl)thiazol-2-yl)amino)benzoic acid (IA65) as a modulator of Orai1. In some embodiments the modulation of Orai1 results in an increase in SOCE in cancer cells while the levels of SOCE in non-cancerous cells are not altered.

### Methods

The present invention provides methods of treating a disease or disorder associated with reduced levels of Ca²⁺ signaling. In some embodiments, the disease or disorder associated with reduced levels of Ca²⁺ signaling is selected from the group consisting of basal cell carcinoma, squamous cell carcinoma, Merkel cell carcinoma, and melanoma. In some embodiments, the method of treating a disease or disorder associated with reduced levels of Ca²⁺ signaling comprises administering a modulator of Orai1, Orai2, or Orai3.

The present invention also provides methods of reducing cancer cell migration, invasion and/or metastasis. In some embodiments, the invention provides a method of reducing cancer cell migration, invasion and/or metastasis by administering a modulator of a Orai1, Orai2, or Orai3. In some embodiments, the invention provides a method of decreasing immune cell activation by administering an activator of Orai1, Orai2, or Orai3.

In some embodiments, the disease or disorder associated with down-regulated or low immune cell activation is cancer. The skilled artisan will understand that treating or preventing cancer in a patient includes, by way of non-limiting examples, killing and destroying a cancer cell, as well as reducing the proliferation of or cell division rate of a cancer cell. The skilled artisan will also understand that a cancer cell can be, by way of non-limiting examples, a primary cancer cell, a cancer stem cell, a metastatic cancer cell. The following are non-limiting examples of cancers that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytoma/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous t-cell lymphoma; esophageal cancer; Ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, Langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, Burkitt; lymphoma, cutaneous t-cell; lymphoma, Hodgkin; lymphoma, non-Hodgkin; lymphoma, primary central nervous system; macroglobulinemia, Waldenstrom; malignant fibrous histiocytoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; paraganglioma; pineal parenchymal tumors of intermediate differentiation; pineoblastoma and supratentorial primitive neuroectodermal tumors; pituitary tumor; plasma cell neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, Ewing family of tumors; sarcoma, Kaposi; sarcoma, soft tissue; sarcoma, uterine; Sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; t-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

It will be appreciated by one of skill in the art, when armed with the present disclosure including the methods detailed herein, that the invention is not limited to treatment of a disease or disorder associated with reduced levels of Ca²⁺ signaling that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with reduced levels of Ca²⁺ signaling, in that a modulator composition, as discussed elsewhere herein, can be administered to a subject prior to the onset of the disease or disorder, thereby preventing the disease or disorder. The preventive methods described herein also include the treatment of a subject that is in remission for the prevention of a recurrence a disease or disorder associated with reduced levels of Ca²⁺ signaling.

One of skill in the art, when armed with the disclosure herein, would appreciate that the prevention of a disease or disorder associated with reduced levels of Ca²⁺ signaling, encompasses administering to a subject a modulator composition as a preventative measure against the development of, or progression of a disease or disorder associated with reduced levels of Ca²⁺ signaling. As more fully discussed elsewhere herein, methods of modulating the level or activity of a gene, or gene product, encompass a wide plethora of techniques for modulating not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases, disorders and pathologies associated with reduced levels of Ca²⁺ signaling, where modulating the level or activity of a gene, or gene product treats or prevents the disease or disorder. Various methods for assessing whether a disease is associated with reduced levels of Ca²⁺ signaling are known in the art. Further, the invention encompasses treatment or prevention of such diseases discovered in the future.

The invention encompasses administration of a modulator of Orai1, Orai2, or Orai3. To practice the methods of the invention; the skilled artisan would understand, based on the disclosure provided herein, how to formulate and administer the appropriate modulator composition to a subject. The present invention is not limited to any particular method of administration or treatment regimen.

One of skill in the art will appreciate that the modulators of the invention can be administered singly or in any combination. Further, the modulators of the invention can be administered singly or in any combination in a temporal sense, in that they may be administered concurrently, or before, and/or after each other. One of ordinary skill in the art will appreciate, based on the disclosure provided herein, that the modulator compositions of the invention can be used to prevent or to treat a disease or disorder associated with reduced levels of Ca²⁺ signaling, and that a modulator composition can be used alone or in any combination with another modulator to achieve a therapeutic result. In various embodiments, any of the modulators of the invention described herein can be administered alone or in combination with other modulators of other molecules associated a disease or disorder associated with reduced levels of Ca²⁺ signaling.

### Modulators

In some embodiments, the present invention provides a method for treating a disease or disorder associated with reduced Ca²⁺ signaling comprising administering a composition to a subject in need thereof, wherein the composition comprises at least one modulator of store-operated Ca²⁺ entry (SOCE) in a subject in need thereof. In some embodiments, the at least one modulator of SOCE is a modulator of Orai1, Orai2, or Orai3. In certain embodiments, the at least one modulator of SOCE is a modulator of Orai1.

For example, In some embodiments, the at least one modulator of Orai1 is 4-((5-Phenyl-4-(trifluoromethyl)thiazol-2-yl)amino)benzoic acid (IA65), or pharmaceutically acceptable salt thereof.

### Small Molecule Modulators

In various embodiments, the modulator is a small molecule. When the inhibitor is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In some embodiments, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

### Nucleic Acid Modulators

In other related aspects, the invention includes an isolated nucleic acid. In some instances, the modulator is an siRNA, shRNA or antisense molecule, which modulates Orai1, Orai2, or Orai3. The invention also encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

In another aspect of the invention, Orai1, Orai2, or Orai3, can be modulated by way of activating, inactivating, releasing, and/or sequestering Orai1, Orai2, or Orai3. As such, modulating the activity of Orai1, Orai2, or Orai3 can be accomplished by using a transdominant positive or negative mutant.

In some embodiments, siRNA or shRNA is used to modulate the level of Orai1, Orai2, or Orai3 protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of modulating levels of, Orai1, Orai2, or Orai3 using RNAi technology.

In another aspect, the invention includes a vector comprising an siRNA or antisense polynucleotide. In some embodiments, the siRNA or antisense polynucleotide is capable of modulating the expression of a target polypeptide, wherein the target polypeptide is selected from the group consisting of p21 and telomerase. The incorporation of a desired polynucleotide into a vector and the choice of vectors is well-known in the art as described in, for example, Sambrook et al. (2012), and in Ausubel et al. (1997).

The siRNA, shRNA, or antisense polynucleotide can be cloned into a number of types of vectors. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

The vector may be obtained by conventional methods known by persons skilled in the art (Sambrook et al., 2012). In a particular embodiment, the vector is a vector useful for transforming animal cells.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin, for example, IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

Antisense molecules and their use for modulating gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby modulating the translation of genes.

The use of antisense methods to modulate the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

### Polypeptide Modulators

In other related aspects, the invention includes an isolated peptide inhibitor that modulates Orai1, Orai2, or Orai3. For example, In some embodiments, the peptide modulator of the invention modulates Orai1, Orai2, or Orai3 directly by binding to Orai1, Orai2, or Orai3 thereby altering the normal functional activity of Orai1, Orai2, or Orai3. In another embodiment, the peptide modulator of the invention modulates Orai1, Orai2, or Orai3 by competing with endogenous Orai1, Orai2, or Orai3. In yet another embodiment, the peptide modulator of the invention modulates the activity of Orai1, Orai2, or Orai3 by acting as a transdominant positive or negative mutant.

The variants of the polypeptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### Antibody Modulator

The invention also contemplates an modulator of Orai1, Orai2, or Orai3 comprising an antibody, or antibody fragment, specific for Orai1, Orai2, or Orai3. That is, the antibody can modulate Orai1, Orai2, or Orai3 to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Activators

In certain embodiments, the invention includes administration of a composition comprising a modulator that increases SOCE, an activator of SOCE. In some embodiments, the activator of SOCE is any compound or molecule that increases the level, activity, or both of Orai1, Orai2, or Orai3. It will be understood by one skilled in the art, based upon the disclosure provided herein, that an increase in the level of Orai1, Orai2, or Orai3 encompasses the increase in Orai1, Orai2, or Orai3 expression, including transcription, translation, or both. The skilled artisan will also appreciate, once armed with the teachings of the present invention, that an increase in the level of a Orai1, Orai2, or Orai3 includes an increase in a Orai1, Orai2, or Orai3 activity (e.g., enzymatic activity, substrate binding activity, etc.). Thus, increasing the level or activity of a Orai1, Orai2, or Orai3 includes, but is not limited to, increasing the amount of Orai1, Orai2, or Orai3 polypeptide, and increasing transcription, translation, or both, of a nucleic acid encoding Orai1, Orai2, or Orai3; and it also includes increasing any activity of Orai1, Orai2, or Orai3 polypeptide as well. In some embodiments, the composition comprises an activator of Orai1.

The increased level of activity of Orai1, Orai2, or Orai3 can be assessed using a wide variety of methods, including those disclosed herein, as well as methods well-known in the art or to be developed in the future. That is, the routineer would appreciate, based upon the disclosure provided herein, that increasing the level or activity of Orai1, Orai2, or Orai3 can be readily assessed using methods that assess the level of a nucleic acid encoding Orai1, Orai2, or Orai3 (e.g., mRNA), the level of a Orai1, Orai2, or Orai3 polypeptide, and/or the level of Orai1, Orai2, or Orai3 activity in a biological sample obtained from a subject.

One of skill in the art will realize that in addition to affecting Orai1, Orai2, or Orai3 directly, diminishing the amount or activity of a molecule that itself diminishes the amount or activity of Orai1 can serve to increase the amount or activity of Orai1, Orai2, or Orai3. Thus, an Orai1, Orai2, or Orai3 activator can include, but should not be construed as being limited to, a chemical compound, a protein, a peptidomimetic, an antibody, a ribozyme, and an antisense nucleic acid molecule. One of skill in the art would readily appreciate, based on the disclosure provided herein, that an Orai1, Orai2, or Orai3 activator encompasses a chemical compound that increases the level, enzymatic activity, or substrate binding activity of Orai1, Orai2, or Orai3. Additionally, an Orai1, Orai2, or Orai3 activator encompasses a chemically modified compound, and derivatives, as is well known to one of skill in the chemical arts.

The Orai1, Orai2, or Orai3 activator compositions employed in the methods of the invention that increase the level or activity (e.g., enzymatic activity, substrate binding activity, etc.) of Orai1, Orai2, or Orai3 include antibodies. The antibodies of the invention include a variety of forms of antibodies including, for example, polyclonal antibodies, monoclonal antibodies, intracellular antibodies (“intrabodies”), Fv, Fab and F(ab)2, single chain antibodies (scFv), heavy chain antibodies (such as camelid antibodies), synthetic antibodies, chimeric antibodies, and humanized antibodies. In some embodiments, the antibody of the invention is an antibody that specifically binds to Orai1, Orai2, or Orai3.

Further, one of skill in the art would, when equipped with this disclosure and the methods exemplified herein, appreciate that an Orai1, Orai2, or Orai3 activator includes such activators as discovered in the future, as can be identified by well-known criteria in the art of pharmacology, such as the physiological results of activation of Orai1, Orai2, or Orai3 as described in detail herein and/or as known in the art. Therefore, the present invention is not limited in any way to any particular Orai1, Orai2, or Orai3 activator as exemplified or disclosed herein; rather, the invention encompasses those activators that would be understood by the routineer to be useful as are known in the art and as are discovered in the future.

Further methods of identifying and producing an Orai1, Orai2, or Orai3 activator are well known to those of ordinary skill in the art, including, but not limited, obtaining an activator from a naturally occurring source (e.g., Streptomyces sp., Pseudomonas sp., Stylotella aurantium, etc.). Alternatively, an Orai1, Orai2, or Orai3 activator can be synthesized chemically. Further, the routineer would appreciate, based upon the teachings provided herein, that an Orai1, Orai2, or Orai3 activator can be obtained from a recombinant organism. Compositions and methods for chemically synthesizing Orai1, Orai2, or Orai3 activators and for obtaining them from natural sources are well known in the art and are described in the art.

One of skill in the art will appreciate that an activator can be administered as a small molecule chemical, a protein, an antibody, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator Orai1, Orai2, or Orai3. (Sambrook et al., 2012, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself diminishes the amount or activity of Orai1, Orai2, or Orai3 can serve to increase the amount or activity of Orai1, Orai2, or Orai3. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity of Orai1, Orai2, or Orai3, thereby increasing the amount or activity of Orai1, Orai2, or Orai3. Contemplated in the present invention are antisense oligonucleotides that are synthesized and provided to the cell by way of methods well known to those of ordinary skill in the art. As an example, an antisense oligonucleotide can be synthesized to be between about 10 and about 100 nucleotides long. In some embodiments, antisense oligonucleotide can be synthesized to be between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of Orai1, Orai2, or Orai3 can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

### Pharmaceutical Compositions and Formulations

The invention also encompasses the use of pharmaceutical compositions to practice the methods of the invention. Such a pharmaceutical composition may consist of at least one modulator composition of the invention or a salt thereof in a form suitable for administration to a subject, or the pharmaceutical composition may comprise at least one modulator composition of the invention or a salt thereof, and one or more pharmaceutically acceptable carriers, one or more additional ingredients, or some combination of these. The compound or conjugate may be present in the pharmaceutical composition in the form of a physiologically acceptable salt, such as in combination with a physiologically acceptable cation or anion, as is well known in the art.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

Although the invention herein is principally directed to the ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, and dogs.

In some embodiments, the compositions utilized in the invention are formulated using one or more pharmaceutically acceptable excipients or carriers. In some embodiments, the pharmaceutical compositions comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In some embodiments, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition utilized in the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In some embodiments, the composition includes an anti-oxidant and a chelating agent that inhibits the degradation of the compound. Exemplary antioxidants for some compounds are BHT, BHA, alpha-tocopherol and ascorbic acid in the range of about 0.01% to 0.3%. In some embodiments, the BHT is in the range of 0.03% to 0.1% by weight by total weight of the composition. In some embodiments, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In some embodiments, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition for use in the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the composition utilized in the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition for use in the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound for use in the invention is from about 1 and 5,000 mg/kg of body weight/per day. One of ordinary skill in the art would be able to study the relevant factors and make the determination regarding the effective amount of the therapeutic compound without undue experimentation.

The invention may be practiced as frequently as several times daily, or it may be practiced less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the composition of the present invention provides for a controlled release of a therapeutic agent, such as a modulator of Orai1, Orai2, or Orai3. In certain instances, controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiment, the composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In some embodiments of the invention, the modulators are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In some embodiments, the invention is practiced in dosages that range from one to five times per day or more. In another embodiment, the invention is practiced in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Routes of administration of include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Suppression of Ca²⁺ Signaling Enhances Melanoma Progression

SOCE is mediated by the members of the Orai and STIM families. Orai channels are Ca²⁺-selective ion channels located at the plasma membrane (PM); STIM is a transmembrane Ca²⁺ sensor associated with the endoplasmic reticulum (ER). STIM responds to ER Ca²⁺ depletion by translocating within the ER towards the PM, and physically associates with PM-localized Orai channels to facilitate Ca′ entry (i.e. SOCE; reviewed in (Soboloff, J., et al, 2012, Nature Reviews Molecular and Cellular Biology, 13:549-565)). Notably, although a growing number of studies have identified links between SOCE magnitude and melanoma progression, results of these studies have been somewhat contradictory. In one prior study, several non-invasive and invasive melanoma cells were investigated, finding an inverse correlation between SOCE and invasiveness (Hooper, R., et al, 2015, Molecular and Cellular Biology, 35:2790-2798). In addition, one randomized, unbiased ribozyme screen for metastasis related genes identified STIM1 as a repressor of melanoma metastasis (Suyama, E., et al, 2004, Journal of Biological Chemistry, 279:38083-38086). However, the SOCE process has been shown to promote melanoma progression in other studies. For example, it has been shown that invasive melanoma relies on enhanced SOCE and AKT activity for growth and survival (Fedida-Metula, S., et al, 2012, Carcinogenesis, 33:740-750; Feldman, B., et al, 2010, Cell Calcium, 47:525-537). Further, invasive melanoma exhibiting activated MAPKs were shown to exhibit higher STIM1 and Orai1 expression (Stanisz, H., et al, 2014, Pigment Cell Melanoma Research, 27:442-453; Sun, J., et al, 2014, Journal of Cell Biology, 207, 535-548; Umemura, M., et al, 2014, PloS One, 9:e89292). Considered collectively, the published evidence fails to support a linear relationship between SOCE and melanoma invasiveness. Given the relationship between UV exposure and melanoma progression, the examples below demonstrate the relationship between UV and SOCE, establishing that UV-induced SOCE suppression plays a critical role in melanoma metastatic progression.

The materials and methods employed are now described.

Cell Culture—SKMEL5, UACC1273, SKMEL2, FS13, WM983 B-Raf-inhibitor resistant (WM983BR), SKMEL28, WM983, UACC257, B16N, B2905A cells were grown in DMEM with 4.5 g/L glucose supplemented with 10% FBS and 1% Gentamycin (Thermo-Fisher Scientific, Waltham, MA) at 37° C. and 5% CO₂.

UV Exposure—Cells were plated on glass coverslips for 24 hours before changing to PBS. Lids were replaced with a thin layer of taut plastic wrap before irradiating with a broadband UV spectrum containing 35% UVA and 65% UVB (with peak emission at 313 nm in the UVB range (De Fabo, E. C., et al, 2004, Cancer Research, 64:6372-6376)). Cells received a total UV dose of 175 J/m² over 90 seconds at a rate of 1.94 W/m². Approximately 80% of the melanoma cells survive this exposure. Cells were passaged 1:10 every 3 days and cultured under growth optimal conditions for the indicated time period (see Cell Culture below) before subsequent experimentation.

SOCE Measurement—Cells grown on glass coverslips were incubated in a cation-safe buffer (107 mM NaCl, 7.2 mM KCl, 1.2 mM MgCl2, 11.5 mM Glucose, 20 mM HEPES-NaOH, 1 mM CaCl₂, pH 7.2) and loaded with Fura2-acetoxymethylester (Fura2-AM; 2 μM) for 30 min at 24° C. as previously described (Go, C. K., et al, 2019, Science Signaling, 12; Hooper, R., et al., 2015, Molecular and Cellular Biology, 35:2790-2798). Cells were washed and allowed to de-esterify for a minimum of 30 min at 24° C. Following de-esterification, cells were treated with 2 μM Thapsigargin (SERCA inhibitor) for 10 minutes prior to imaging. Ca²⁺ measurements were taken using a Leica DMI 6000B fluorescence microscope controlled by Slidebook software (Intelligent Imaging Innovations, Denver, CO). Fluorescence emission at 505 nm was monitored in response to excitation at alternating 340 nm and 380 nm wavelengths at a frequency of 0.67 Hz; intracellular Ca²⁺ measurements are shown at 340/380 nm ratios obtained from groups (2-45 for patient samples, 35-45 for established cell lines) of single cells.

RT-qPCR—Cells were grown to 70% confluency and collected as cell pellets prior to RNA extraction (Invitrogen, Waltham, MA). Following RNA extraction, 500 ng RNA was converted to cDNA using ezDNAse™ and SuperScript® IV Reverse Transcriptase Assay Kit (Invitrogen, Waltham, MA) and diluted 1:10 for working concentration of 50 ng per reaction. Housekeeping 18S ribosomal RNA (18S) and genes of interest were used for each reaction (500 nM) mixed with Powerhouse SYBR® green (Applied Biosystems, Bedford, MA) and analyzed using QuantStudio8 software (Applied Biosystems, Bedford, MA).

Proliferation Assays—500 cells/well were plated on a 96 well plate for the indicated time point and treated with 1:10 WST-1 for 1 hour at 37° C., 5% CO2. Absorbance was measured using GloMax® plate reader at 450 nm.

Transwell Migration Assays—Transwell migration assays were performed as previously described (Hooper, R., et al., 2015, Molecular & Cellular Biology, 35:2790-2798). Briefly, Matrigel® transwell chambers (Corning, Bedford, MA) were hydrated with serum-free OptiMEM for 2 hours (37° C., 5% CO2) prior to the addition of 2,500 cells/well. Growth optimal media was placed on the bottom of the chamber prior to incubation (20 hours; 37° C., 5% CO₂). After incubation, media was removed, and edges were cleaned before methanol fixation and staining with crystal violet (0.5%, 2 min). Images were taken by EVOS cell imager and quantified on ImageJ.

Generating stable Orai1-WT-CFP and Orai1-E106A-CFP cell lines—B16N and WM983 cells were transfected with either pIRES-Neo Orai1-WT-CFP or pIRES-Neo Orai1-E106A-CFP vector via electroporation using Gene Pulser Xcell™ (Biorad). Cells were cultured for 1 week under growth optimal conditions before selection with G418 for 2 weeks. Cell sorting was used to isolate the top 5% of the CFP expressing cells CFP before growth and cloning. Clones were then expanded and screened for CFP fluorescence intensity and SOCE on Leica DMI 6000B fluorescence microscope controlled by Slidebook software.

Viability Assays—500,000 cells were plated in 24 well plates overnight in the absence or presence of 2 μM IA65. Cells were then trypsinized with 0.25% Trypsin-EDTA for 10 minutes at 37° C. Following, cells were treated with 0.4% Trypan Blue and placed in a Bio-Rad slidereader and the number of stained vs. unstained cells compared.

In vivo subcutaneous injections—B16N cells were counted using a hemocytometer and 1×10⁶ cells in 200 μl sterile PBS were injected subcutaneously on the right flank of syngeneic C57BL6 mice. Mice were monitored for 14 days and then sacrificed followed by full body dissection and histopathological analysis. Tissues were fixed in 10% Neutral-Buffered Formalin (pH 6.8-7.2) followed by H&E staining and Aperio® Imagescope software (Leica Biosystems; Buffalo Grove, IL) scan for peritoneal wall invasion.

RNA sequencing analysis—Raw RNA-Seq data were processed using the subread algorithm established in (Liao, Y., et al, 2014, Bioinformatics, 30:923-930). Preprocessed data files from the samples were subject to RNA sequencing data analysis using Rsubread package (Liao, Y., et al, 2019, Nucleic Acids Research, 47:e47-e47). Genome indices were built using the buildindex function in Rsubread package. RNA transcripts from each sample were mapped to the mouse genome reference consortium build 38 (GRCm38) genome. Alignment of the sample reads to the reference genome was performed using the align function within the Rsubread package. feature Counts function within the Rsubread package was used to summarize the data to integer-based, gene-level read counts. Read counts generated from the pipeline were annotated using mm10 annotation. Differential expression analysis of the read counts was performed using the DESeq2 package (Anders, S. & Huber, W., 2010, Genome Biology, 11:R106-R106). Differentially expressed genes generated from the DESeq2 analysis were subjected to pathway analysis using Ingenuity Pathway Analysis tool (Ingenuity® Inc, Redwood city, CA) with default settings.

Migration Assay—Cells were plated on glass coverslips (Ibidi, Fitchburg, WI) overnight to form a complete monolayer before “wounding” with a p20 pipette tip. Coverslips were placed in an incubation chamber (37° C.; 5% CO2) and monitored by confocal microscopy (Leica SP8 Laser Scanning Microscope). Z-stack brightfield images were obtained every 20 minutes for 20 hours; the % of the wound that was refilled over time was calculated using ImageJ.

Transfection of mCherry-PFO-D4H—WM983 and UACC257 human melanoma cells were grown to 50% confluency on before transfecting with PFO D4H-mcherry via electroporation using Gene Pulser Xcell™ (Biorad, Hercules, CA). Cells were then plated on glass coverslips under growth optimal conditions before capturing images by confocal microscopy (Leica SP8 Laser Scanning Microscope; Buffalo Grove, IL). Fluorescence intensity was measured using Leica LASX software.

Isolation of Cells from Patient Surgical Explants—Tissue was incubated in DMEM containing Liberase® TL (Roche Applied Science; 37° C., 30 min). Following incubation, tissue was cut using a sharp razor and dissociated through 50 μM Medicon screens (Becton Dickinson Biosciences, San Jose, CA; 2 min). Tissue samples were resuspended in DFD media (DMEM, 20% FBS; 500 ug/ml DNAse I, Sigma, St. Louis, MO) and passed through an 18-gauge syringe to break up clumps of cells before filtration (70 μm, Corning, Glendale, AZ, centrifugation and resuspension in DMEM (2% FBS; 1% Gentamycin) for 3 days before use.

Glucose Uptake Assay—WM983 and UACC257 treated as described were cultured under growth optimal conditions parallel to unmanipulated control cells. Cells were seeded at density of 15,000 cells/well in 96 well plates. Glucose uptake was measured using the Promega® Glucose Uptake-Glo Assay (Madison, WI); luminescence was recorded using 0.3-1 second integration using a Tecan INFINITE® M1000 PRO monochromator-based microplate reader.

Seahorse Assays—Oxygen Consumption Rate (OCR) and Extracellular Acidification rate (ECAR) were measured using an Agilent Seahorse® XF96 (Wilmington, DE). B16N, WM983 and UACC257 cells treated as described were cultured under growth optimal conditions parallel to unmanipulated control cells. 15,000 cells/well were seeded in Seahorse® 96 well cell culture plates for all experiments. Basal OCR was measured in media supplemented with 1 mM pyruvate, 2 mM glutamine, and 10 mM glucose followed by the sequential addition of oligomycin (1.5 μM), FCCP (1 μM) and rotenone/antimycin-A (1 μM) to perform the mitochondrial stress test. Basal ECAR was measured in media supplemented with 2 mM glutamine followed by the sequential addition of glucose (10 mM), oligomycin (1 μM) and 2-deoxy-d-glucose (2-DG: 50 mM) to perform the glycolysis stress test. Results were quantified using Wave software (Agilent Technologies, Santa Clara, CA).

Materials—All cell lines were acquired from American Type Culture Collection (ATCC; Manassas, VA). Media was purchased from Corning and Gibco (Waltham, MA). TAK475 and DNAse I were purchased from Sigma-Aldrich (St. Louis, MO). Compactin and BTP2 were purchased from Tocris Bio-Techne Corporation (Minneapolis, MN). OSMI1 was purchased from Millipore Sigma (Burlington, MA). Matrigel® Transwell Chambers were purchased from Corning (Waltham, MA). Liberase® TL was purchased from Roche Applied Science (Indianapolis, IN). 50 μM Medicons and the medimachine were purchased from Becton Dickinson Biosciences (San Jose, CA). 8 well u-slides migration plates were purchased from Ibidi (Fitchburg, WI). Fura2-AM was purchased from Thermo-Scientific (Waltham, MA). Glucose Uptake Kit was purchased from Promega (Madison, WI), Lactate Production Kit was purchased from Sigma-Aldrich (St. Louis, MO). High fat mouse chow was purchased from Harlan labs (Frederick, MD). Orai1-WT-CFP and Orai1-E106A-CFP were generated as previously described (Zhou et al, 2009); the mCherry-PFO-D4 gene was synthesized by Integrated DNA Technologies (Coralville, IA) and combined via PCR into an mCherry-C1 backbone (Addgene). PFO-D4H was constructed via site-directed mutagenesis.

The Results are now discussed.

### UV Suppresses SOCE and Enhances Invasion in Melanoma

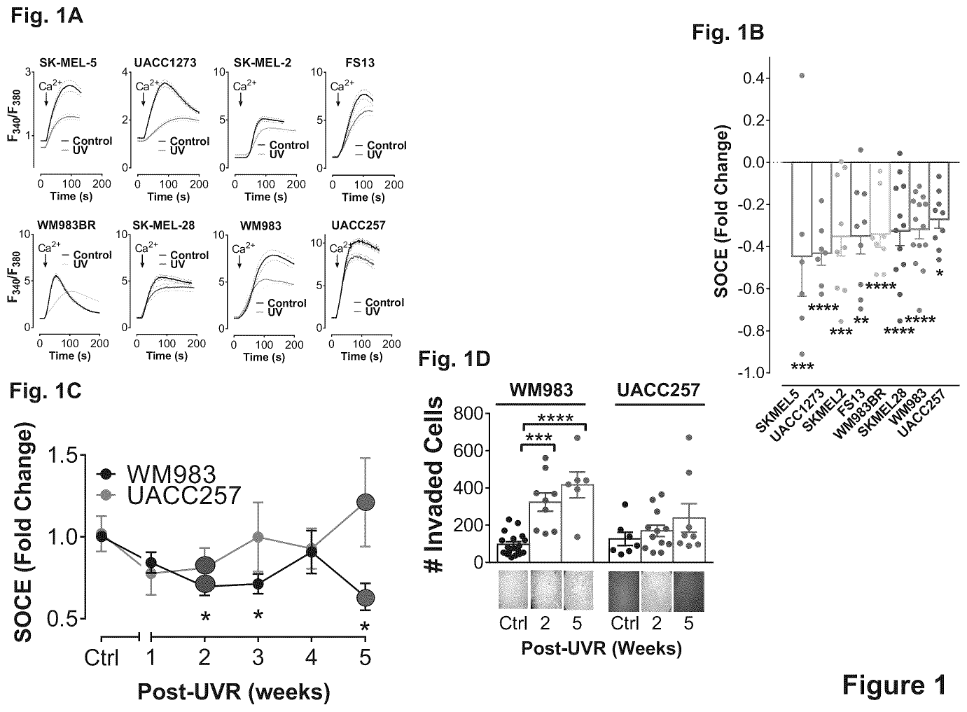

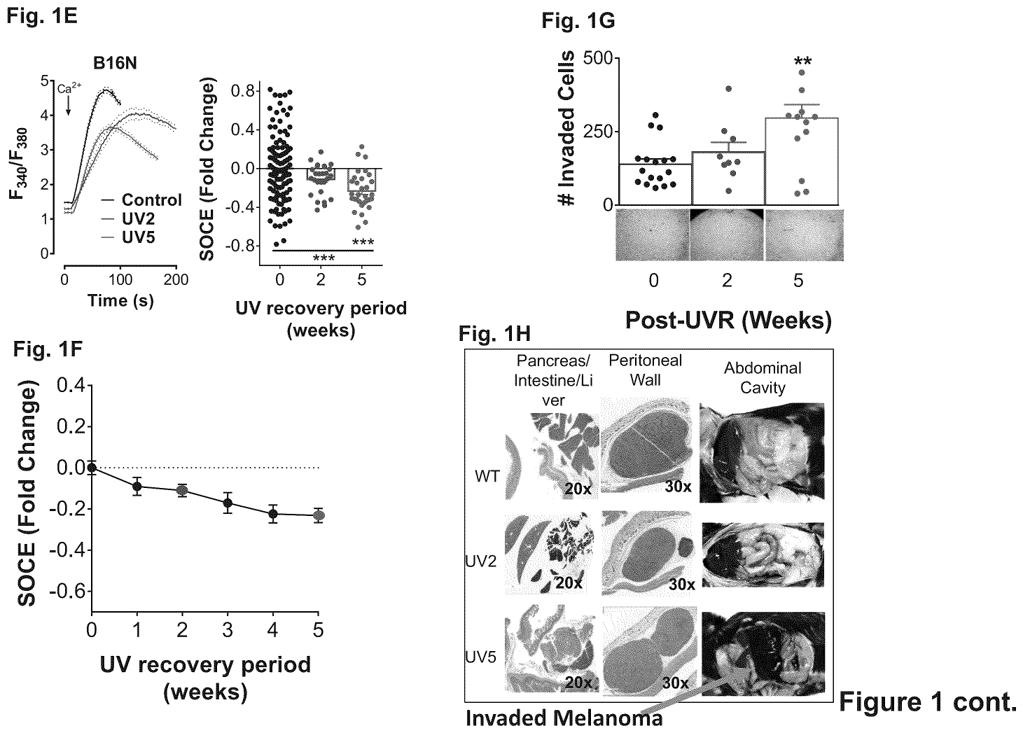

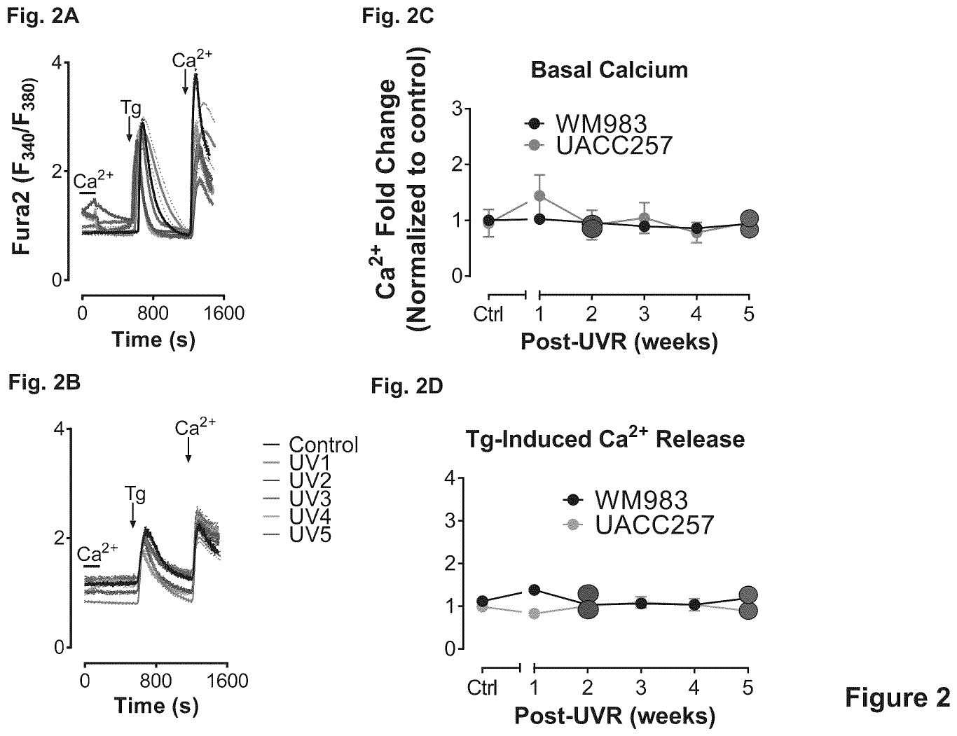

The effect of UV on SOCE in melanoma was assessed in a panel of established non-metastatic human melanoma cell lines containing a range of different common driver mutations common to melanoma (SKMEL5, UACC1273, SKMEL2, FS13, SKMEL28, UACC257)(Bairoch, A., 2018, J. Biomol. Tech., 29:25-38)) and ((WM983, WM983BR)(Herlyn, M., 2021, The Wistar Institute)). Cells were exposed to UV (175 J/m²), then allowed to recover for 24 hours. To induce SOCE, cells were treated with the Sarco/Endoplasmic Reticulum Calcium ATPase (SERCA) pump inhibitor thapsigargin (Tg; 2 μM) for 10 minutes to deplete the ER of Ca²⁺ prior to the addition of 1 mM Ca²⁺. Addition of exogenous Ca²⁺ induced SOCE in all cell lines, although variable SOCE amplitudes were observed amongst the different cell lines (FIG. 1A). Prior exposure to UV suppressed SOCE in all cell lines tested 24 hours after a single UV exposure (FIGS. 1A-1B); no relationship to driver mutations in the different cell lines could be detected. To determine if this was a transient or stable effect, basal calcium, Tg-induced Ca2+ release, and SOCE were tracked on a weekly basis for 5 weeks in WM983 and UACC257 cells after a single exposure to UV (FIG. 1C). Although there was a modest change in basal and Tg-induced Ca2+ release in WM983 and UACC257 cells after 5 weeks, WM983 cells exhibited substantial SOCE suppression at 2-3 weeks after UV exposure (FIG. 1C and FIG. 2). WM983 cells exhibited significant SOCE suppression at 2-3 weeks after UV exposure followed by a second, steeper suppression of SOCE between 4 and 5 weeks whereas no significant changes in SOCE were observed in UACC257 cells in response to UV exposure. To determine if changes in UV-dependent changes in SOCE were specific or reflective of a general shift in Ca²⁺ homeostasis, both basal and ER Ca²⁺ content were also measured in WM983 and UACC257 cells recovering from UV exposure (FIG. 2). No significant changes were observed at any timepoint in either WM983 or UACC257 cells, leading to the conclusion that UV exposure alters SOCE, but not basal or ER Ca²⁺ content.

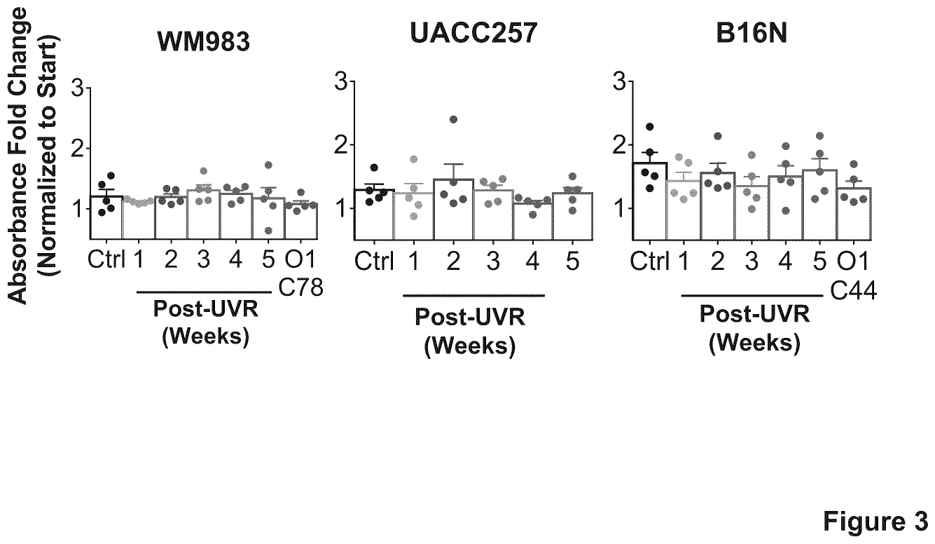

To determine if differences in the stability of UV-induced SOCE suppression in WM983 and UACC257 cells correlated with invasiveness, transwell invasion assays were performed on non-irradiated cells or cells exposed to UV and allowed to recover for either 2 or 5 weeks (hereafter termed UV2 or UV5, respectively; FIG. 1D). Notably, whereas several-fold increases in the number of invasive UV2 and UV5 WM983 were observed, no UV-induced changes in UACC257 invasiveness were observed. To determine if increased invasiveness might reflect UV-dependent effects on cell viability and/or proliferation, WST-1 proliferation assays were performed (FIGS. 2A-2B and FIG. 3), with no UV-dependent differences found in WM983 or UACC257 cells. These data demonstrate a direct correlation between SOCE suppression and invasive behavior in UV-induced WM983, but not UACC257 melanoma progression, along with an intriguing delay between UV exposure and reprogramming of invasive responses.

To determine if UV-induced SOCE suppression and invasiveness would also occur in vivo, the study was extended to B16N melanoma cells, with the ultimate goal of assessing metastasis in immunocompetent C57Bl/6 mice. Paralleling results in human melanoma cells, UV exposure led to SOCE suppression after a 5-week incubation (FIG. 1E-F), with a similar increase in invasion through Matrigel® in a transwell invasion assay in vitro (FIG. 1G), with no significant effect on cell proliferation (FIG. 3). In complementary work, untreated, UV2 or UV5 B16N cells were injected s.c. in syngeneic C57BL/6 mice and allowed to grow for 2 weeks before being euthanized and assessing metastasis by pathology. Consistent with prior studies (Overwijk & Restifo, 2001, Curr. Protoc. Immunol., Chapter 20; Valle, E. F., et al, 1992, Clin. Exp. Metastasis, 10:419-429), in 26 out of 28 mice injected with untreated B16N cells, no evidence of metastasis was found (Table 1). In contrast, over half of the mice (8/15) injected with UV2 B16N cells had detectable metastases, as did 79% (19/24) of mice injected with UV5 B16N cells (Table 1). Interestingly, for all B16N models, infiltration was observed throughout the peritoneum, but not in the lungs (FIG. 1H; Table 1). While it is more typical for melanoma to metastasize to the lungs, these data nevertheless demonstrate a potent UV-dependent shift towards invasive behavior.

TABLE 1
Metastasis in mice after introduction of subcutaneous B16N tumors

| | | | Abdominal | |
|---|---|---|---|---|
| | | Invasion | Metastases | |
| | Total | through | Formed from | |
| Mouse | Mouse | Peritoneal | Subcutaneous | |
| Condition | Number | Wall**** | Tumors** | Sites of Mets Found |
| Control | 28 | 2/28 (7.14%) | 0/28 (0%) | Peritoneum (2 mice) |
| UV2 | 15 | 8/15 (53.3%) | 2/15 (13.3%) | Peritoneum (2 mice) |
| | | | | Liver (2 mice) |
| | | | | Pancreas (2 mice) |
| | | | | Kidney (1 mouse) |
| UV5 | 24 | 19/24 (79.1%) | 9/24 (37.5%) | Peritoneum (9 mice) |
| | | | | LN (8 mice) |
| | | | | Pancreas (4 mice) |
| | | | | Intestine (3 mice) |
| | | | | Liver (8 mice) |
| | | | | Mesentery (1 mouse) |
| | | | | Spleen (1 mouse) |
| O1C44 | 14 | 13/14 (92.9%) | 3/14 (21.4%) | Peritoneum (3 mice) |
| | | | | LN (3 mice) |
| | | | | Kidney (3 mice) |
| | | | | Intestine (2 mice) |
| | | | | Spleen (1 mouse) |
| | | | | Liver |

Significant differences in invasiveness through the peritoneal wall and into the abdomen was determined by the chi squared test. ** P<0.01; ****P<0.0001

### Melanoma Progression Mediated by SOCE Suppression Exhibits a “Goldilocks Effect”.

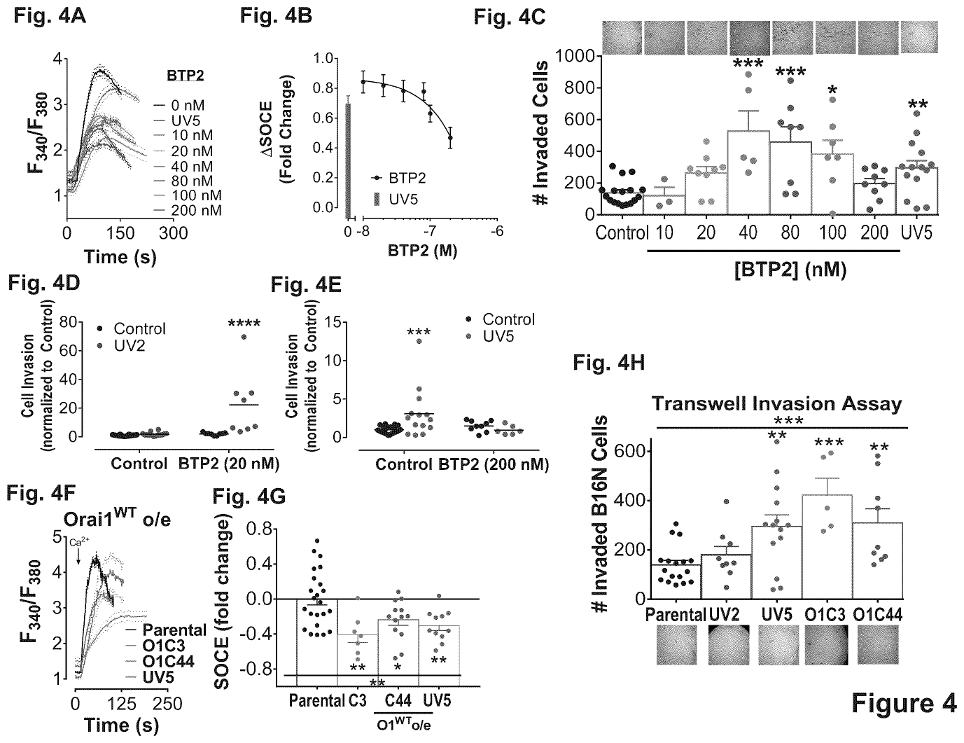

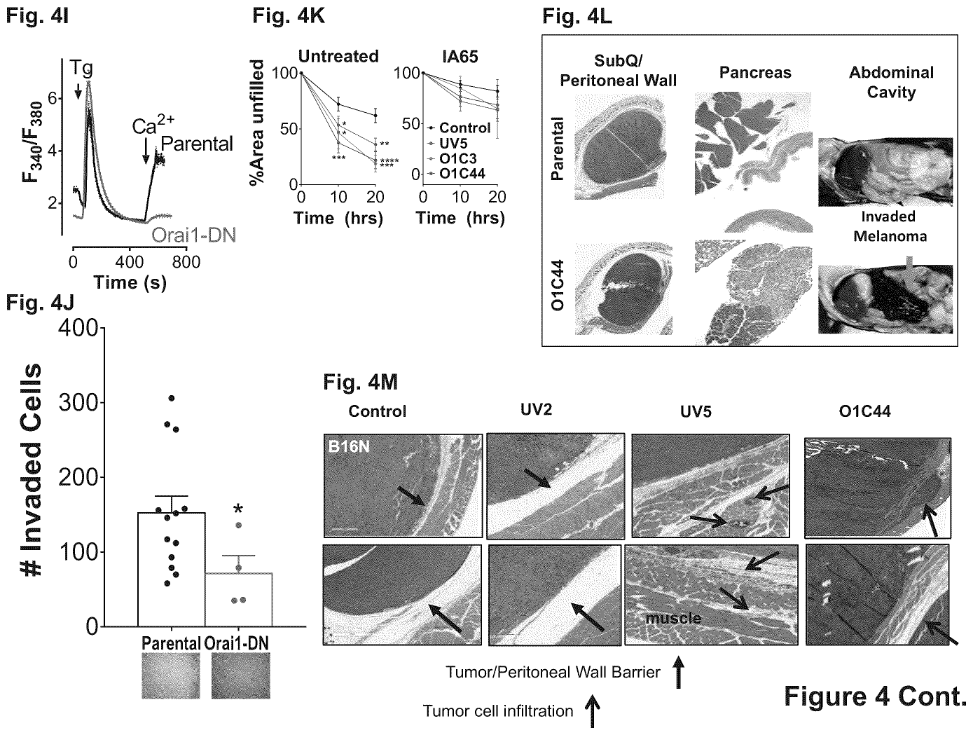

UV has a wide range of short- and long-term effects on cell function (Coelho, S. G., et al, 2009, Journal of Investigative Dermatology Symposium Proceedings, 14:32-35), only one of which is SOCE suppression. To more directly establish a relationship between suppressed SOCE and invasiveness, SOCE was suppressed in B16N cells using 3,5-Bis(trifluoromethyl)pyrazole (BTP2), a well-established inhibitor of both Orai1-mediated SOCE and TRPC-mediated Ca²⁺ entry (He, L. P., et al, 2005, Journal of Biological Chemistry, 280:10997-11006; Ishikawa, J., et al, 2003, Journal of Immunology, 170:4441-4449; Schleifer, H., et al, 2012, British Journal of Pharmacology, 167:1712-1722; Zitt, C., et al, 2004, Journal of Biological Chemistry, 279-12427-12437). Titration of BTP2 at a concentration range of 20 to 100 nM caused levels of SOCE suppression comparable to UV (FIGS. 4A-4B), and additionally stimulated several-fold increases in invasiveness through transwell invasion assays (FIG. 4C). The effect on invasiveness was lost at higher BTP2 concentrations (e.g., 200 nM) suggesting an “Goldilocks effect”. Similar observations were made in WM983 cells (Figure EV2). To further probe this concept, non-invasive UV2 B16N melanoma cells were treated with BTP2 (20 nM), a concentration that had no effect on invasiveness in parental B16N cells (FIG. 4D). Increased invasiveness was observed, suggesting that SOCE was insufficiently suppressed in both cases to drive an increase in invasiveness through Matrigel®. The contribution of SOCE to the enhanced invasiveness of B16N UV5 cells was then assessed by measuring invasiveness in the presence of BTP2 (200 nM; FIG. 4E). This completely blocked invasiveness, indicating that a minimum amount of SOCE is needed to support invasive behavior, consistent with the proposed “Goldilocks effect”, at a concentration of BTP2 well below toxic levels (Ishikawa, J., et al., 2003, Journal of Immunology, 170:4441-4449; Zitt, C., et al., 2004, Journal of Biological Chemistry, 279:12427-12437).

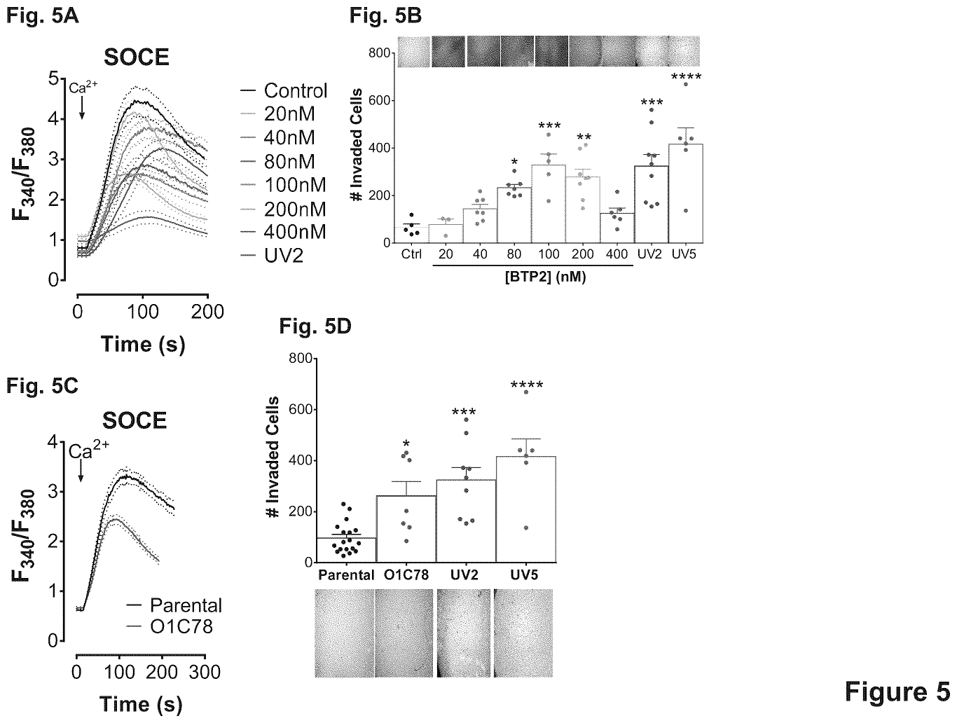

Validation was achieved by genetic manipulation of SOCE. Overexpression of Orai1 has been reported to suppress SOCE through stoichiometric imbalance with STIM1 (Hoover, P. J., & Lewis, R., S., 2011, Proceedings of the National Academy of Sciences of the USA, 108:13299-13304; Soboloff, J., et al, 2006, Journal of Biological Chemistry, 281:20661-20665). A series of B16N cell lines stably expressing Orai1 were generated and screened to determine the level of SOCE suppression. Two Orai1-overexpressing clones that exhibited SOCE suppression at levels similar to UV5 B16N cells were identified and designated O1C3 and O1C44 (FIGS. 4F-4G). In both cases, these cells demonstrated enhanced invasion through Matrigel® in a transwell invasion assay, to levels similar to those seen with UV5 B16N cells (FIG. 4H). Similar observations were made in an Orai1-overexpressing clone of WM983 cells (FIG. 5).

The impact of a dominant negative Orai1 mutant (Orai1-E106A-CFP; Orai1-DN; (Prakriya, M., et al, 2006, Nature, 443:230-233; Vig, M., et al, 2006, Current Biology, 16:2073-2079; Yeromin, A. V., et al, 2006, Nature, 443:226-229)) previously shown to block invasion (Sun, J., et al., 2014, Journal of Cell Biology, 207:535-548; Umemura, M., et al., 2014, PloS One, 9:e89292) was assessed. B16N cells stably expressing Orai1-DN exhibited both ablated SOCE (FIG. 4I) and severely diminished invasion in a transwell invasion assay (FIG. 4J), revealing a non-linear relationship between SOCE and invasiveness in melanoma, in which partial suppression enhances invasion, but SOCE blockade abrogates it.

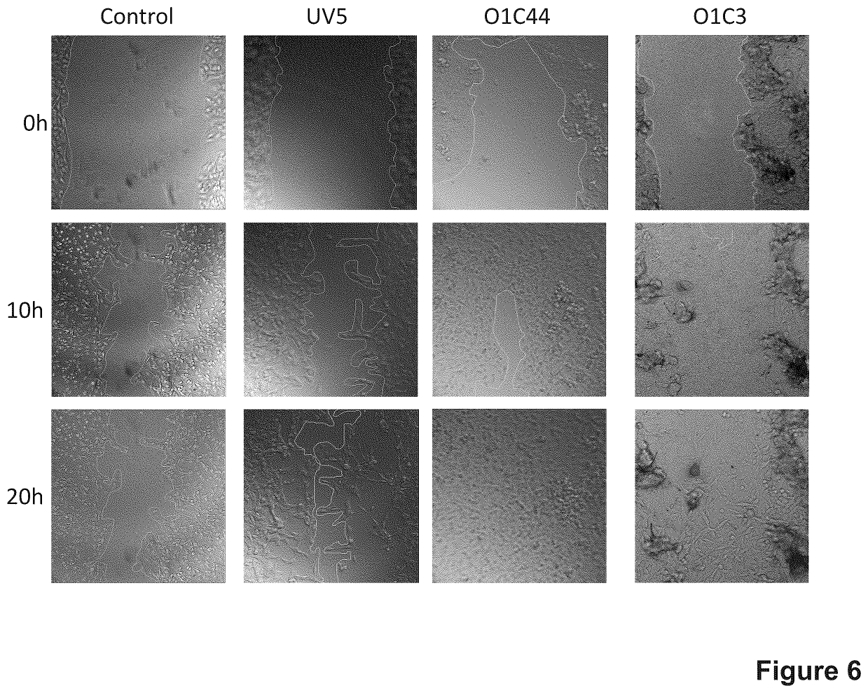

An important property of invasion is migration, which is regulated in part by transient changes in Ca²⁺ levels that facilitate filipodia formation, lamellipodia attachment, and activation of myosin light chain kinases (MLCKs) that control movement (reviewed in (Gross, M., et al, 2020b, Advances in Cancer Research, 148:233-317)). To assess the effect of SOCE on migratory potential, O1C44 and UV5 B16N cells were seeded as monolayers, grown overnight, scratched to create a wound, then imaged every 20 minutes for 20 hours. Both O1C44 and UV5 B16N cells exhibited greater migratory behavior compared to control cells (FIG. 2K and FIG. 6). SOCE suppression in the absence of UV created a more profound effect on migration, with Orai1-overexpressing cells nearly completely filling the wound 10 hours after initial scratch. Considered together, these data demonstrate SOCE suppression as a driving force behind migration and a mediator of UV-dependent enhancement of melanoma invasion.

To determine if SOCE suppression is sufficient to drive invasiveness in syngeneic mice, Orai1-overexpressing B16N cells were injected s.c. and metastasis was assessed by pathology. Similar to mice injected with UV-irradiated cells, extensive metastasis was observed throughout the peritoneal cavity in 13 out of 14 mice (Table 1; FIG. 4L). Although less common for melanoma, there is evidence showing that several metastatic cancers have been known to penetrate the peritoneal cavity through the peritoneal wall, a process commonly referred to as intraperitoneal carcinomatosis (IC) (Desai J. P., 2020, Cancer, Peritoneal Metastasis, StatPearls Publishing; Lee, E. S., et al, 2014, Obstetrics & Gynecology Science, 57:240-243; McBride, M. & Calhoun, S., 2019, Journal of Radiology Case Reports, 13:28-37; Mikula-Pietrasik, J., et al, 2018, Cellular and Molecular Life Sciences, 75:509-525). Further, high resolution images of the peritoneal wall reveal no untreated B16N cells, but significant penetration of UV-irradiated or Orai1-expressing B16N cells (FIGS. 2M-2N). The 3 mice containing O1DN tumors showed less than 0.1 mm² of total invasion, even less than the 2 control mice, exhibiting peritoneal wall invasion (FIG. 2N). Considered collectively, these data reveal that modest SOCE suppression promotes metastasis from an s.c. melanoma, while SOCE ablation has minimal or no effect.

### SOCE Recovery Inhibits Invasion

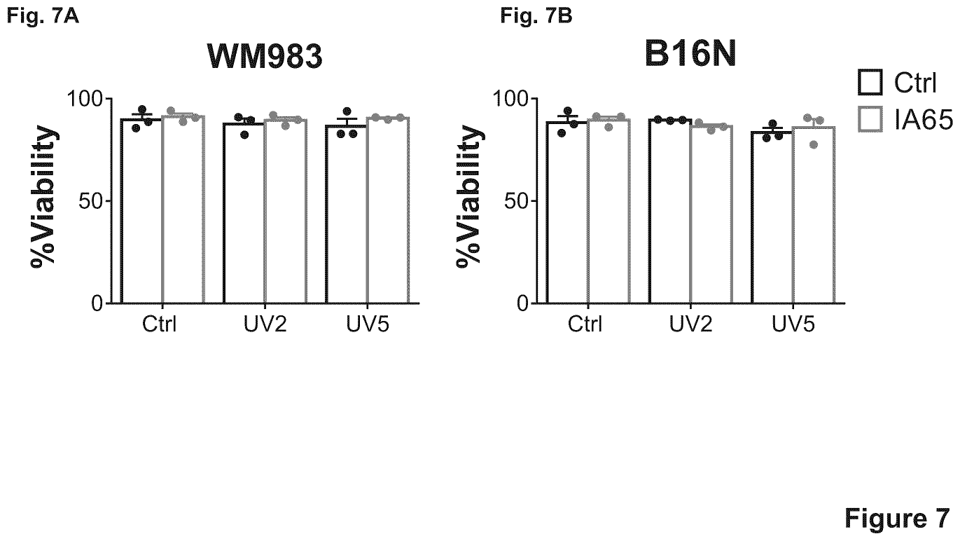

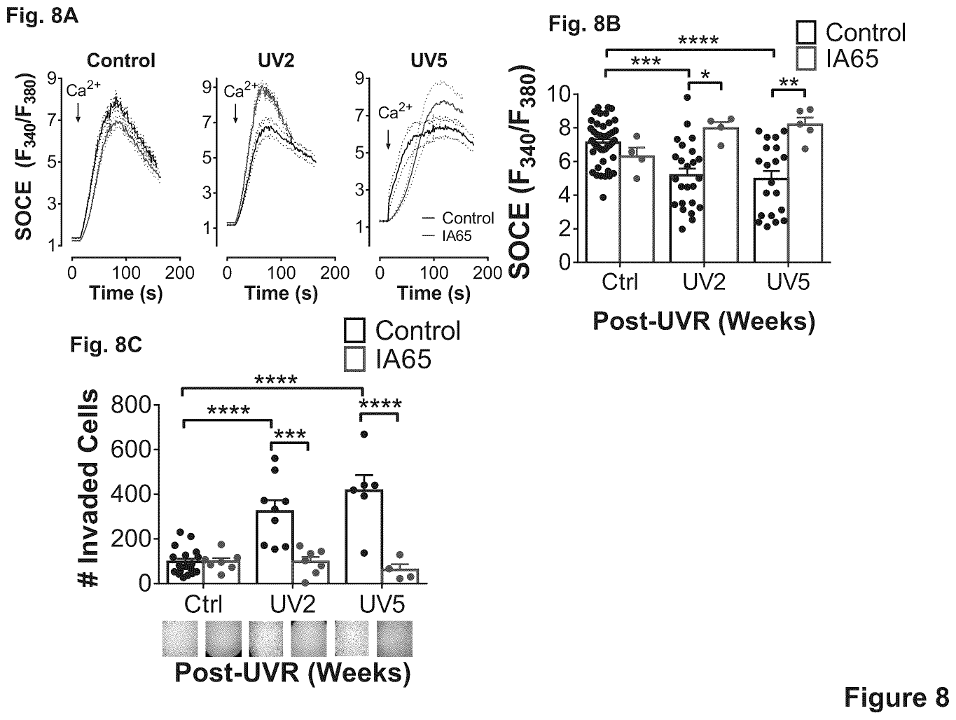

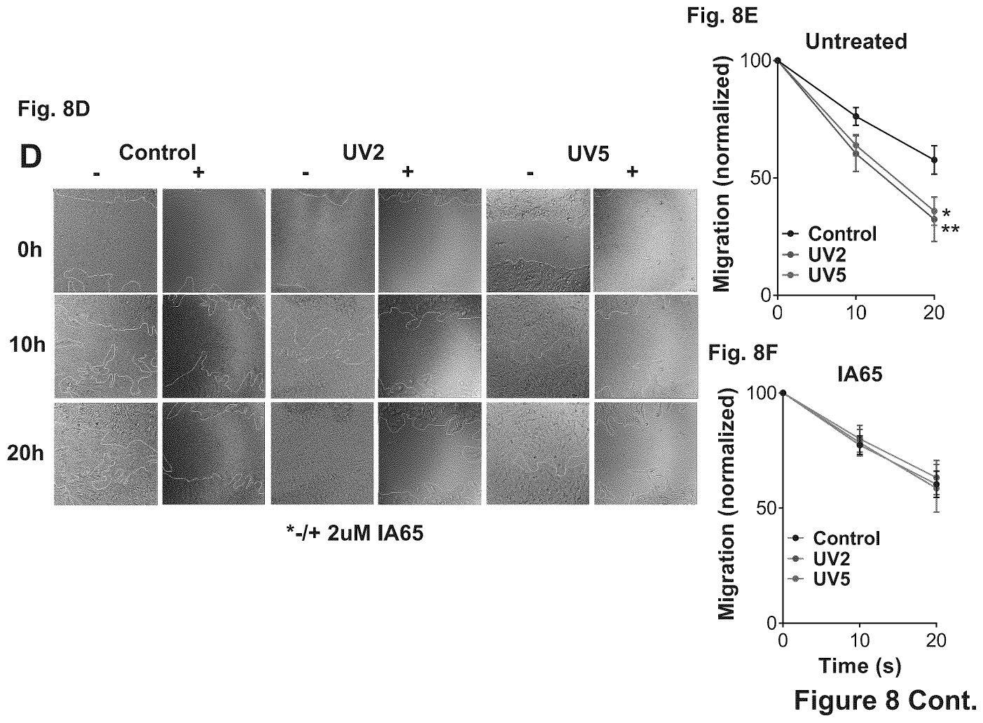

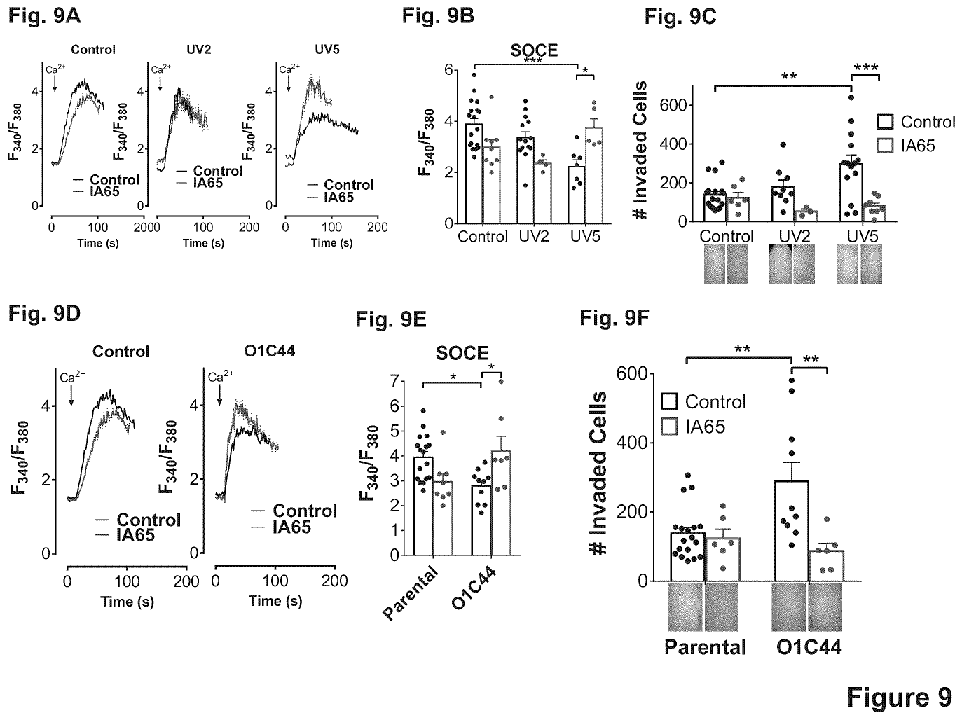

To further establish the concept that SOCE suppression contributes to UV-induced invasiveness 4-((5-Phenyl-4-(trifluoromethyl)thiazol-2-yl)amino)benzoic acid (IA65), a potentiator of Orai-1 and SOCE (Azimi, I., et al, 2020, ACS Pharmacology & Translational Science, 3:135-147; Zhang, X., et al, 2020, Cell Calcium, 91:102281), was used. IA65 had no effect on cell viability (FIG. 7) or on SOCE in untreated WM983 cells, but strikingly reversed UV-induced SOCE suppression (FIGS. 8A and 8B). Notably, WM983 cells treated with IA65 failed to invade through Matrigel® in a transwell assay even after exposure to UV (FIG. 8C) and did not have UV-induced enhanced cell migration (FIG. 8D-8F). Similar observations of the effect of IA65 on SOCE, migration, and invasion were made in B16N cells (FIG. 9). Collectively, these observations indicate that SOCE suppression is a required component of UV-induced increases in invasiveness in melanoma cells.

### Identification of the Mechanism of UV-Induced SOCE Suppression.

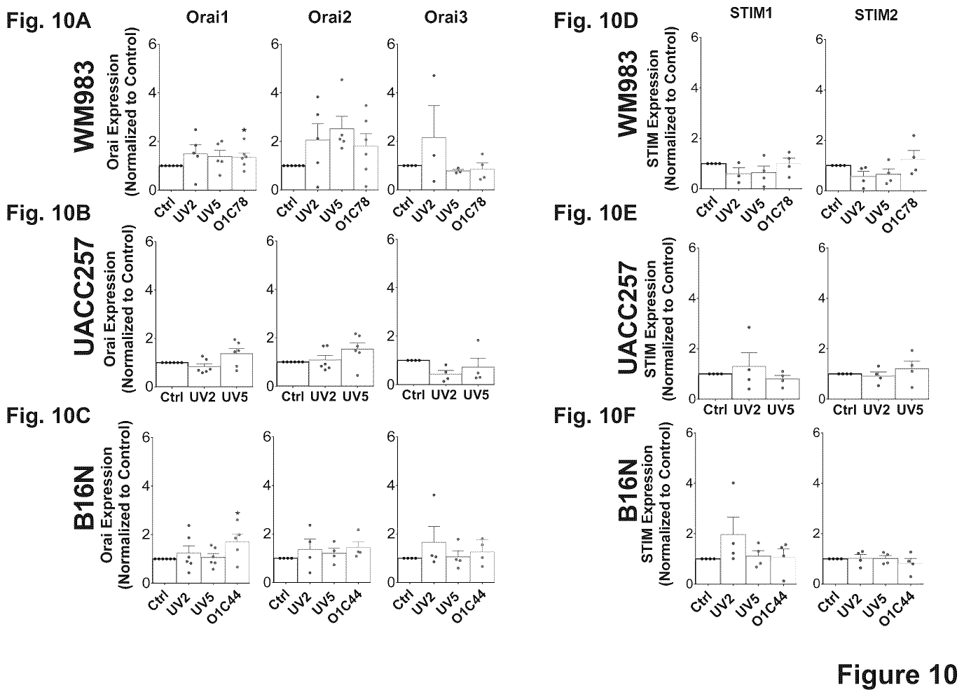

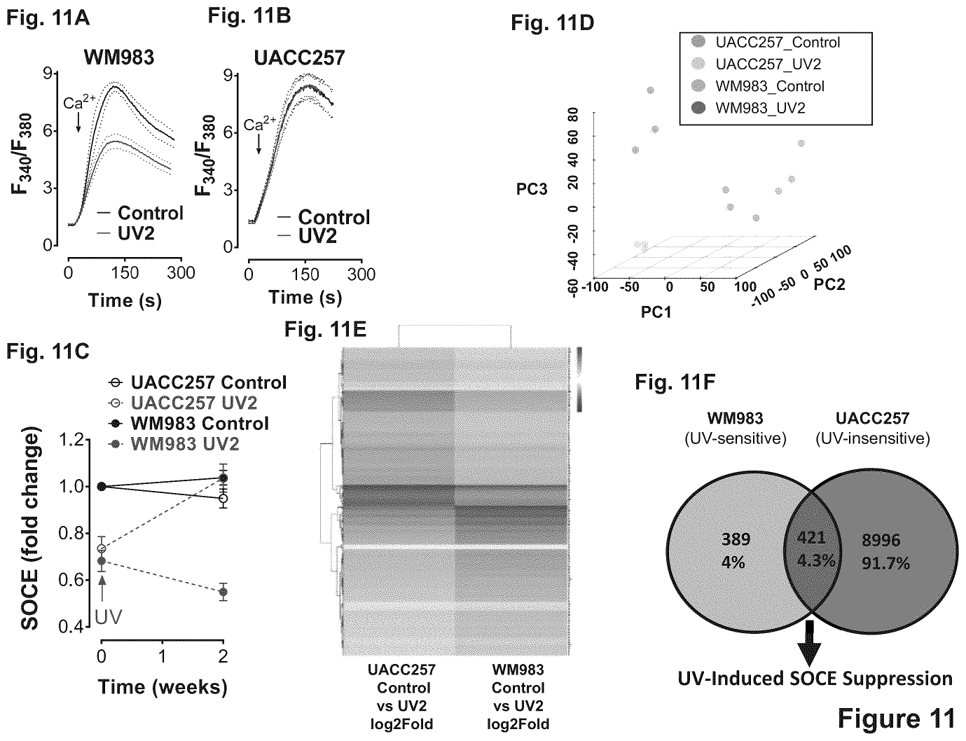

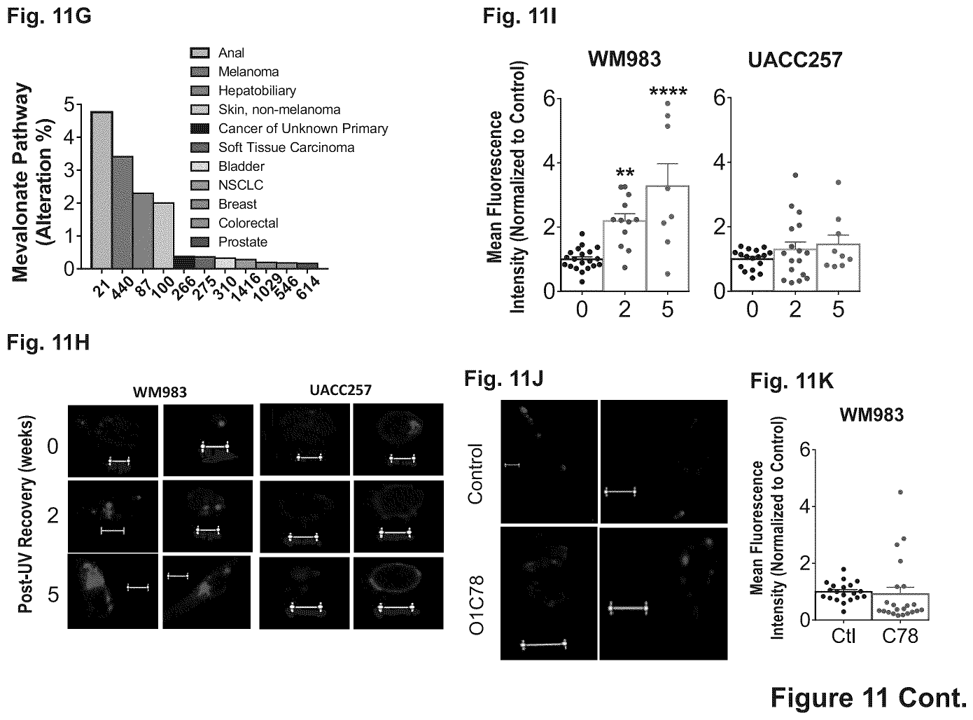

To determine the mechanism of UV-induced SOCE suppression, first the level of expression of the 5 members of the STIM and Orai families was measured by RT-qPCR (FIG. 10). No significant UV-induced changes in the expression of Orai genes were observed in WM983 or B16N cells (FIGS. 10A and 8C), although significant UV-induced increases in Orai1 expression did occur in UACC257 (p=0.0441; FIG. 10B). Since no significant UV-induced changes in SOCE were observed in UACC257 cells, the biological significance of this observation is unclear. Both O1C78 WM983 and O1C44 B16N cells exhibited a modest, but statistically significant increase in Orai1 expression (FIGS. 10A and 10C). No significant, changes in the expression of either STIM1 or STIM2 were observed in WM983, UACC257 or B16N cells (FIG. 10D-10F). Since no decreases in STIM or Orai expression were observed in UV5 cells, these data do not provide an explanation for UV-induced SOCE suppression. To gain mechanistic insight into UV suppression of SOCE, RNAseq experiments were performed, taking advantage of the observed differences in SOCE suppression in WM983 and UACC257 cells 2 weeks after UV exposure, comparing each cell model to non-irradiated cells (see FIG. 1C; FIGS. 11A-11C). Principal component analysis (PCA) revealed both minimal replicate variability and the existence of 4 discrete groups, with UV having distinct effects in WM983 and UACC257 cells (FIG. 11D). Comparison of the genes changed in these cell types reveals marked expression differences in the effect of UV on UACC257 and WM983 cells (FIG. 11E). To identify those genes whose expression was altered by UV that could contribute to SOCE suppression, Venn analysis was performed, comparing the genes whose expression was significantly altered (p<0.01, q<0.01) by UV exposure in WM983, but not UACC257 cells (FIG. 11F), followed by Ingenuity Pathway Analysis (IPA) on this group of 421 genes. The pathway whose expression was most altered was the superpathway of cholesterol (CHL) biosynthesis (also known as the mevalonate pathway; Table 2) with 8 out of 28 genes upregulated (Table 3). CHL has been previously identified as an endogenous inhibitor of SOCE due to a direct interaction with Orai1 (Derler, I., et al, 2016, Science Signaling, 9:ra10). Further, the mevalonate pathway has been to shown to contribute to melanoma progression in vivo (Pencheva, N., et al, 2014, Cell, 156:986-1001). Finally, Cancer Genome Atlas (TCGA) analysis determined if the mevalonate pathway was similarly altered in clinical tumor samples (FIG. 11G). Notably, melanoma was the second most likely tumor-type to exhibit mevalonate pathway dysregulation (3.4%). Therefore, possibility that enhanced CHL biosynthetic pathways contributes to SOCE suppression and UV-induced melanoma invasiveness and metastasis was assessed.

TABLE 2
IPA Analysis of RNAseq of WM983 and UACC257 from FIG. 11 showing top
ranked upregulated pathways

| Ingenuity Canonical Pathways | −log(p-value) | Ratio | Molecules |
|---|---|---|---|
| Superpathway of Cholesterol | 7.76 | 0.286 | MVD, FDPS, IDI1, LSS, HMGCR, |
| Biosynthesis | | | HMGCS1, GGPS1 |
| Superpathway of | 6.56 | 0.353 | MVD, FDPS, IDI1, HMGCR, |
| Geranylgeranyldiphosphate | | | HMGCS1, GGPS1 |
| Biosynthesis I (via Mevalonate | | | |
| Ethanol Degradation IV | 5.46 | 0.24 | TYRP1, ALDH1L2, ALDH1A3, |
| | | | ACSS2, CAT, ALDH3B1 |
| Trans, trans-farnesyl Diphosphate | 4.3 | 0.6 | FDPS, IDI1, GGPS1 |
| Biosynthesis | | | |
| Mevalonate Pathway I | 4.25 | 0.308 | MVD, IDI1, HMGCR, HMGCS1 |
| Oxidative Ethanol Degradation III | 3.38 | 0.19 | ALDH1L2, ALDH1A3, ACSS2, ALDH3B1 |
| Oleate Biosynthesis II (Animals) | 2.89 | 0.231 | FADS2, ALDH6A1, FADS1 |
| Geranylgeranyldiphosphate | 2.76 | 0.5 | FDPS, GGPS1 |
| Biosynthesis | | | |
| Ethanol Degradation II | 2.43 | 0.108 | ALDH1L2, ALDH1A3, ACSS2, ALDH3B1 |
| Histamine Degradation | 2.39 | 0.158 | ALDH1L2, ALDH1A3, ALDH3B1 |
| Antigen Presentation Pathway | 2.39 | 0.105 | HLA-A, HLA-F, TAP2, HLA-DPA1 |
| Fatty Acid α-oxidation | 2.21 | 0.136 | ALDH1L2, ALDH1A3, ALDH3B1 |
| Putrescine Degradation III | 2.15 | 0.13 | ALDH1L2, ALDH1A3, ALDH3B1 |
| Molecular Mechanisms of Cancer | 1.69 | 0.033 | ALDH1L2, ALDH1A3, ALDH3B1, RAC2, |
| | | | LRP5, GSK3A, RALBP1, CCND1, PAK1, |
| | | | CCNE, CDK11B, SMO, PRKAR1B, |
| | | | APH1B, ARHGEF9, BCL2L11 |

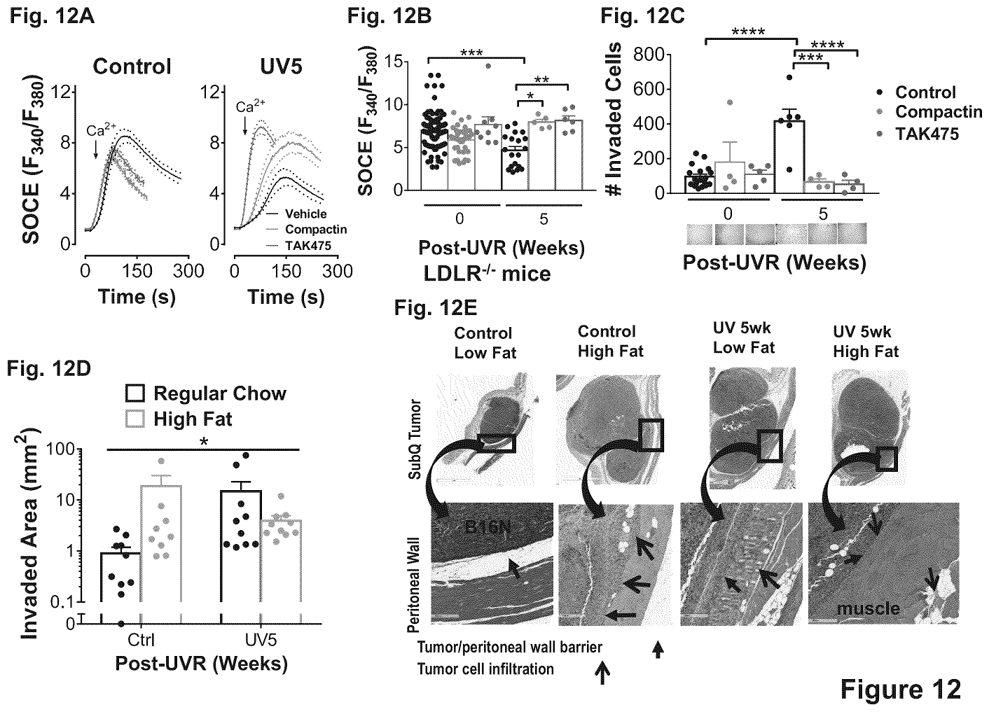

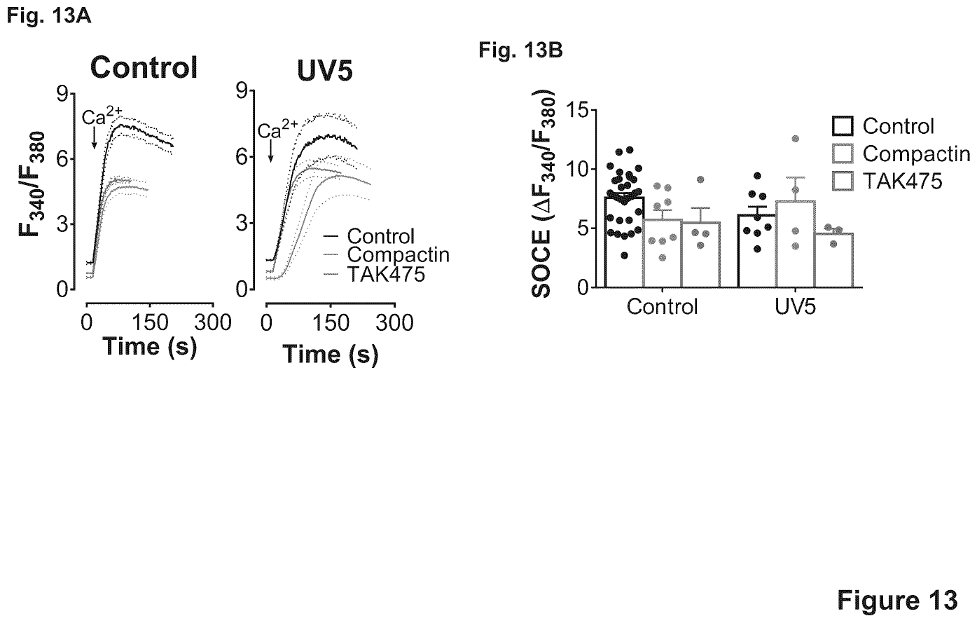

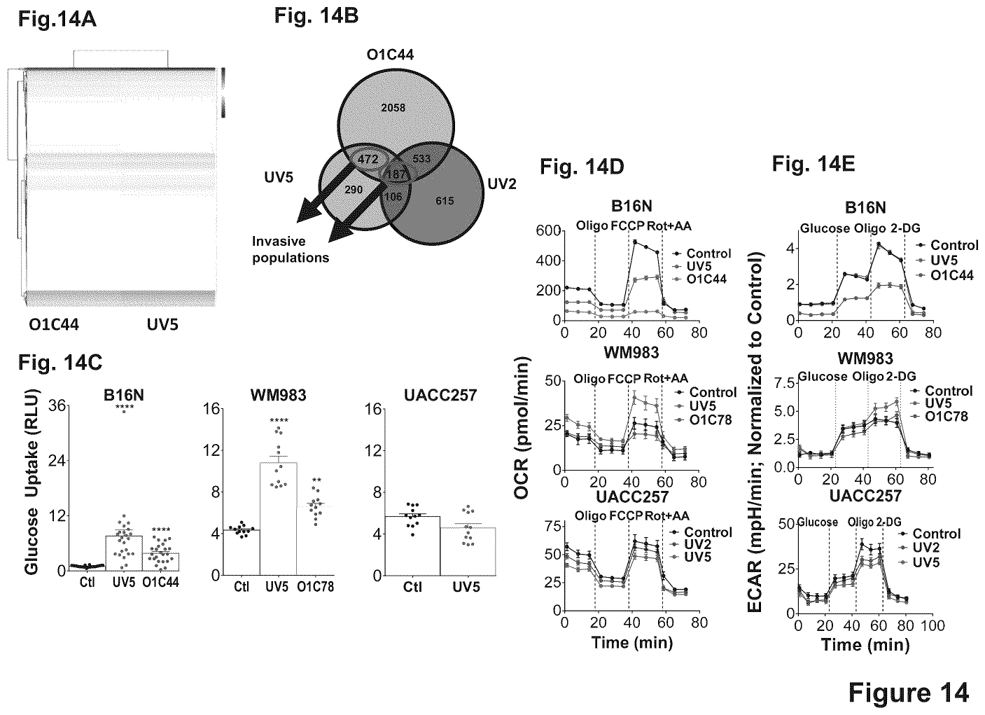

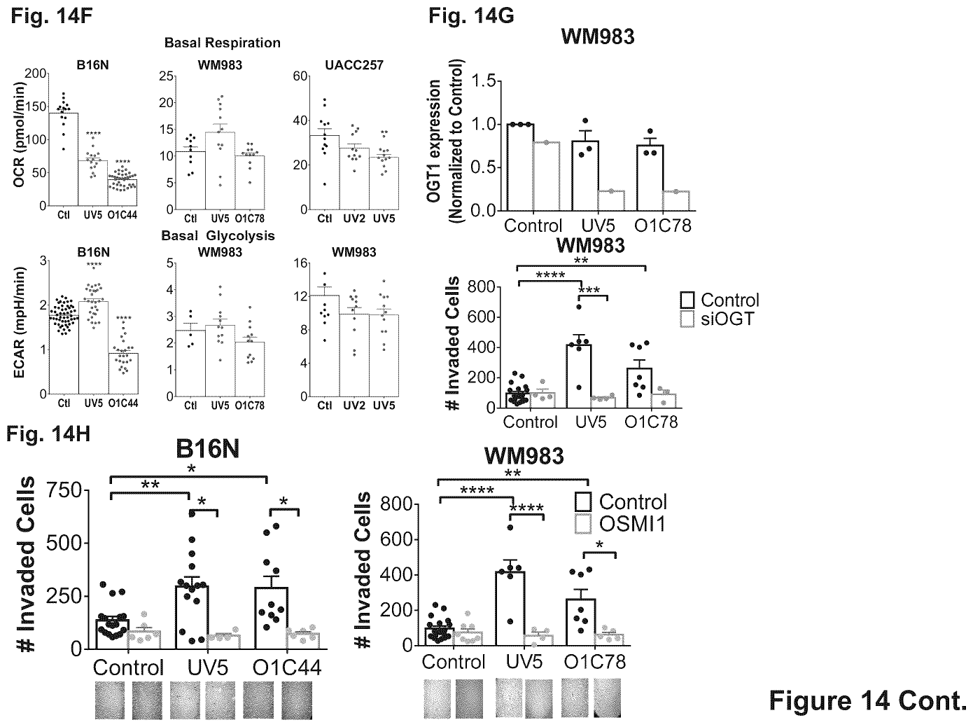

To determine if UV treatment increased CHL content, designed CHL sensors based on Perfringolysin O (PFO) toxin), a toxin with CHL-binding activity (Maekawa, M., 2017, Sensors (Basel, Switzerland), 17:504)) were designed and utilized. Briefly, the CHL-binding portion of the PFO toxin was mutated to increase affinity with a D434S point mutation within the D4 domain (D4H), tagged with mCherry and transfected into WM983 and UACC257 cells (FIGS. 10H and 10I). UV markedly increased mCherry expression in WM983 in a time-dependent manner, but did not do so in UACC257 cells (FIGS. 10H and 10I), which correlates with the effect of UV on SOCE on these 2 cell types (FIG. 1C). Analogous experiments were done in an Orai1 overexpressing clone of WM983 cells (O1C78) and showed no significant change in mCherry expression, revealing that cholesterol upregulation is upstream of SOCE suppression and is likely not bidirectional (FIGS. 11J and 11K). To assess if UV-induced CHL production contributes to UV-induced SOCE suppression, cells were treated with either the HMG-CoA reductase inhibitor compactin (a statin drug) or the squalene synthase (SS) inhibitor TAK475, which targets a downstream step in the CHL biosynthesis pathway. While neither agent significantly affected SOCE in untreated WM983 cells, SOCE in UV5 WM983 cells was markedly enhanced after a 24 hr treatment with either agent (FIGS. 12A-12B). Further, when these same CHL inhibitor drugs were given to non-SOCE suppressed UACC257 UV5 cells, no change occurred in SOCE (FIG. 13). Additionally, both drugs abrogated UV-induced increases in melanoma invasiveness through Matrigel® on a transwell assay, while having no effect on invasiveness in untreated cells (FIG. 14C).

Finally, to assess the contribution of CHL on melanoma metastasis in vivo, the metastasis of parental and UV5 B16N cells grown for 5 weeks post-irradiation before implantation s.c. in Ldlr^{-/-} mice was analyzed (FIGS. 12D and 12E). Ldlr^{-/-} mice have normal CHL levels when fed regular chow but exhibit dramatic increases when fed high fat diet (Huszar, D., et al, 2000, Arteriosclerosis, Thrombosis, and Vascular Biology; Towler, D. A., et al, 1998, Journal of Biological Chemistry, 273:30427-30434; Tsuchiya, K., et al, 2012, Cell Metabolism, 15:372-381). High fat chow led to marked increases in peritoneal wall invasion and IC metastasis (FIGS. 12D and 12E) independent of UV, strongly suggesting that enhanced CHL biosynthesis is sufficient to mimic UV-induced melanoma metastasis. These observations support a model in which UV drives melanoma metastasis due to SOCE suppression mediated by increased CHL biosynthesis.

TABLE 3
Expression ratios of genes within the superpathway of cholesterol
biosynthesis pathway between UV-irradiated WM983 and UACC257 cells
Gene Significantly Altered by UV-Induced SOCE Suppression

| | | Expr |
|---|---|---|
| Abbreviation | Name | Log Ratio |
| FDPS | Farnesyl Diphosphate Synthase | 1.21 |
| GGPS1 | Geranylgeranyl Pyrophosphate Synthase 1 | 1.174 |
| HMGCR | 3-Hydroxy-3-Methylglutaryl Coenzyme | 1.272 |
| | A Reductase | |
| HMGCS1 | 3-Hydroxy-3-Methylglutaryl Coenzyme | 1.276 |
| | A Synthase 1 | |
| IDI1 | Isopentyl Diphosphate Delta Isomerase 1 | 1.367 |
| LSS | Lanosterol Synthase | 1.07 |
| MSMO1 | Methylsterol Monooxygenase 1 | 1.664 |
| MVD | Diphosphomevalonate Decarboxylase | 1.28 |
| | (Mevalonate) | |

Genes Not Significantly Altered by UV-Induced SOCE Suppression

| | | Expr |
|---|---|---|
| Abbreviation | Name | Log Ratio |
| ACAT | Acetyl Co-A: Cholesterol Acetyltransferase | 0.300562 |
| MVK | Mevalonate Kinase | 0.848137 |
| PMVK | Phosphomevalonate Kinase | −0.0864 |
| FDFT | Farnesyl Diphosphate Farnesyl Transferase/ | 0.592592 |
| | Squalene Synthase | |
| SQLE | Squalene Monooxygenase/Epoxidase | 0.696172 |
| CYP51A1 | Sterol 14-Demethylase | 0.634207 |
| TM7SF2 | D14-Sterol Reductase | 0.419114 |
| HSD11B1 | 3b-Hydroxy-4a-Methylcholestenecarboxylate | No value |
| | 3-Dehydrogenase (Decaboxylating) | reported |
| HSD17B7 | 3-Keto-Steroid Reductase/17b-Hydroxysteroid | 0.525395 |
| | Dehydrogenase 7 | |
| EBP | Cholestenol D-Isomerase | 0.057375 |
| SC5D/SC5DL | Lathosterol Oxidase | 0.824012 |
| DHCR7 | 7-Dehydrocholesterol Reductase | 0.935822 |
| DHCR24 | D24-Sterol Reductase | −0.40312 |
| CYP11A1 | Cytochome P450 Family 11 Subfamily | 0 |
| | A Member 1 | |
| PGGT1B | Geranylgeranyltransferase | 0.395243 |
| DHDDS | Dehydrodolichol Diphosphate Synthase | 0.069556 |
| | Subunit | |
| PDSS1 | Decaprenyl Diphosphate Synthase Subunit 1 | −0.59921 |
| PDSS2 | Decaprenyl Diphosphate Synthase Subunit 2 | 0.175392 |
| COQ2 | 4-Hydroxybenzoate Decaprenyltransferase | 0.166566 |
| COX10 | Cytochrome C Oxidase Assembly | −0.76864 |
| | Factor Heme A:Farnesyl Transferase | |

### SOCE Suppression Drives a Metabolic Shift Towards Anabolic Pathways

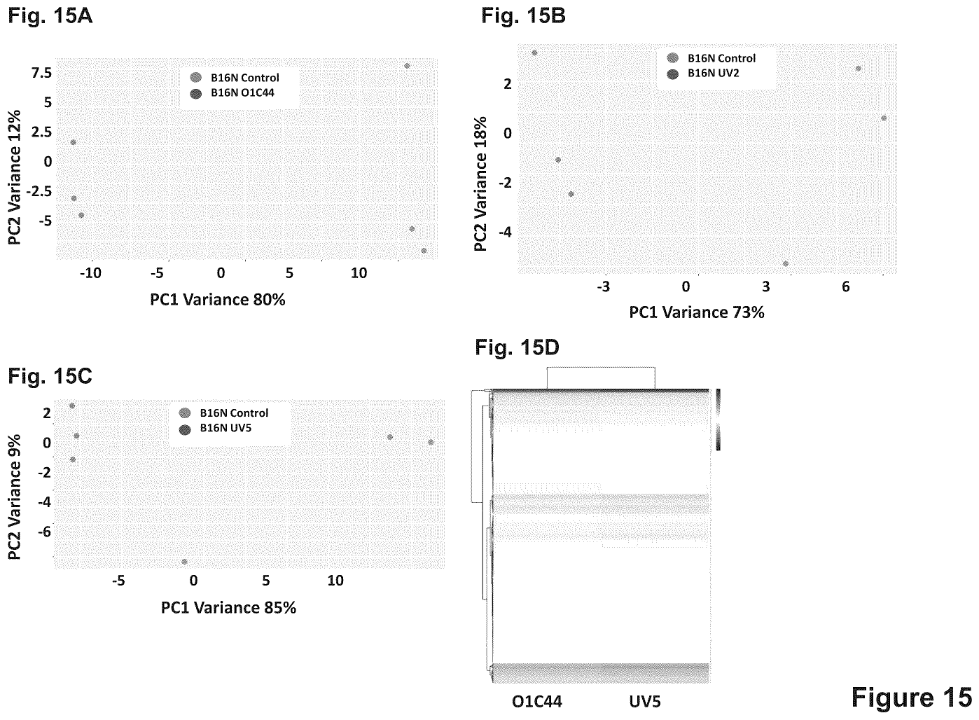

In an effort RNAseq analysis of untreated, UV2, UV5, and O1C44 B16N cells to determine how SOCE suppression contribute to invasive behavior in melanoma (FIGS. 14A and 14B; FIG. 7). PCA revealed minimal replicate variability and significant differences from control for each experimental condition (FIG. 15) and marked differences in expression patterns between each of the 3 groups (FIG. 14A; FIG. 10). Venn analysis comparing the genes whose expression was significantly altered (p<0.01, q<0.01) in UV5, O1C44 and/or UV2 was performed to identify genes associated with both SOCE suppression and invasiveness. The exclusion of UV2 in one group was performed because these cells did not exhibit SOCE suppression (FIGS. 1E,1F) and were not invasive through a transwell assay (FIG. 1G) but did exhibit metastatic properties in a small subset of mice (FIGS. 4L and 4M; Table 1). There were 187 genes changed that were common to all 3 groups, including multiple genes associated with epithelial-mesenchymal transition (EMT), melanocyte dedifferentiation, metabolism and cell survival (Table 4). If the non-SOCE suppressed UV2 cells are excluded, there were 472 genes changed. With the exception of dedifferentiation, the number of pathways changed in each category increased when UV2 was excluded, particularly as related to control of glucose uptake and metabolism (Table 4). Dysregulation of both Ca²⁺ signals and metabolism in the context of cancer has been extensively reported (Reviewed in (Dejos, C., et al, 2020, Frontiers in Cell Developmental Biology, 8:573747; Prevarskaya, N., et al, 2018, Physiological Reviews, 98:559-621)); since all gene changes were observed in O1C44 cells in which SOCE is suppressed, metabolic function was assessed.

Analysis of glucose uptake revealed significant many-fold increases in the highly invasive UV5 and O1C44 B16N cells, UV5 and O1C78 WM983 cells, although not in UV5 UACC257 cells (FIG. 14C). Further, analysis of oxygen consumption rate (OCR) and extracellular acidification rate (ECAR) revealed only modest UV or Orai1-dependent changes in OCR in B16N, WM983, and UACC257 cells (FIG. 14D) and either decreased or unchanged ECAR (FIG. 14E). Measurements of Basal Respiration and Basal Glycolysis show either decreased or unchanged aerobic respiration of glycolytic activity across both human and murine cells (FIG. 14F). Importantly, these changes in ECAR and OCR did not correlate well with the invasiveness phenotypes of these cells, implying the highly elevated levels of imported glucose were being used for alternative purposes than glycolysis.

TABLE 4
IPA Analysis showing key pathways altered from RNAseq

| Target | | | −log(p- | #genes |
|---|---|---|---|---|
| groups | Category | Pathway | value) | affected* |
| O1C44, | Cancer Biology | Molecular Mechanisms of | 2.52 | 9/453 |
| UV5 | | Cancer | | |
| and | | Wnt-Beta Catenin Signaling | 2.99 | 6/174 |
| UV2 | | ERBB Signaling | 3.42 | 5/94 |
| | Invasion | Epithelial-Mesenchymal | 3.52 | 7/195 |
| | | Transition | | |
| | | Epithelial Adherens | 4.05 | 7/160 |
| | | Junction Signaling | | |
| | | Gap Junction Signaling | 3.31 | 7/211 |
| | | Integrin Signaling | 2.54 | 6/214 |
| | De- | Melanocyte Development | 4.39 | 6/96 |
| | differentiation | and Pigmentation Signaling | | |
| | Metabolism | Acetyl-CoA Biosynthesis | 2.94 | 2/8 |
| | | from Citrate | | |
| | Cell survival | Ferroptosis | 4.11 | 7/156 |
| O1C44, | Cancer Biology | Molecular Mechanisms of | 1.77 | 14/400 |
| UV5 | | Cancer | | |
| not | Metabolism | Aryl Hydrocarbon Signaling | 5.6 | 13/143 |
| UV2 | | Xenobiotic Metabolism of | 4.58 | 9/85 |
| | | Aryl Hydrocarbon Signaling | | |
| | | Pentose Phosphate Pathway | 2.32 | 2/6 |
| | | (Non-oxidative) Glucose | | |
| | | Metabolism Disorder | 3.78 | 69/472** |
| | | Impaired Glucose Tolerance | 2.85 | 10/472** |
| | Cell Survival | ERK/MAPK Signaling | 2.36 | 10/202 |
| | | Sirtuin Signaling | 2.1 | 12/291 |
| | | Myc-Mediated Apoptosis | 1.87 | 4/50 |
| | | Signaling | | |
| | Invasion/Cell- | Gap Junction Signaling | 1.95 | 9/198 |
| | Cell Contact | FAK Signaling | 1.9 | 6/104 |
| | | Integrin Signaling | 1.76 | 9/213 |

Although most glucose is normally processed through glycolysis, glucose can also be shunted down an alternative biosynthetic route, the hexosamine biosynthetic pathway (HBP). In normal cells, this represents roughly 2-5% of glucose consumption, however, cancer cells have been shown to upregulate use of the HBP (Akella, N. M., et al, 2019, BMC Biology, 17:52; Marshall, S., et al, 1991, Journal of Biological Chemistry, 266:4706-4712). Elevated HBP increases uridine diphosphate-N-acetylglucosamine (UDP-GlcNAc) production, which ultimately drives post-transcriptional O-GlcNAcylation mediated by the enzyme O-linked N-acetylglucosamine (GlcNAc) transferase (OGT), promoting EMT, proliferation, cell survival, and transcription (Akella, N. M., et al., 2019, BMC Biology, 17:52). RT-qPCR analysis of OGT gene expression revealed either unchanged or decreased levels of OGT in response to UV exposure or SOCE suppression (FIG. 14G) which implies that changes in HBP activity are likely caused by increased substrate and OGT activity. To determine whether elevated OGT activity contributes to the increased invasiveness of cells with depressed SOCE, cells were treated with siRNA targeting OGT (siOGT; FIG. 14G) and a specific and selective pharmacological inhibitor of OGT, OSM1 (Barkovskaya, A., et al, 2019, Scientific Reports, 9:5670-5670). These inhibitors reduced invasion across all SOCE-suppressed conditions in both WM983 and B16N cells (FIG. 14H). These observations reveal a critical role for OGT activity in the observed increase in invasiveness due to SOCE suppression in melanoma cells.

Patient Surgical Explants have Low SOCE and a Similar Pharmacological Profile to Melanoma Cell Lines

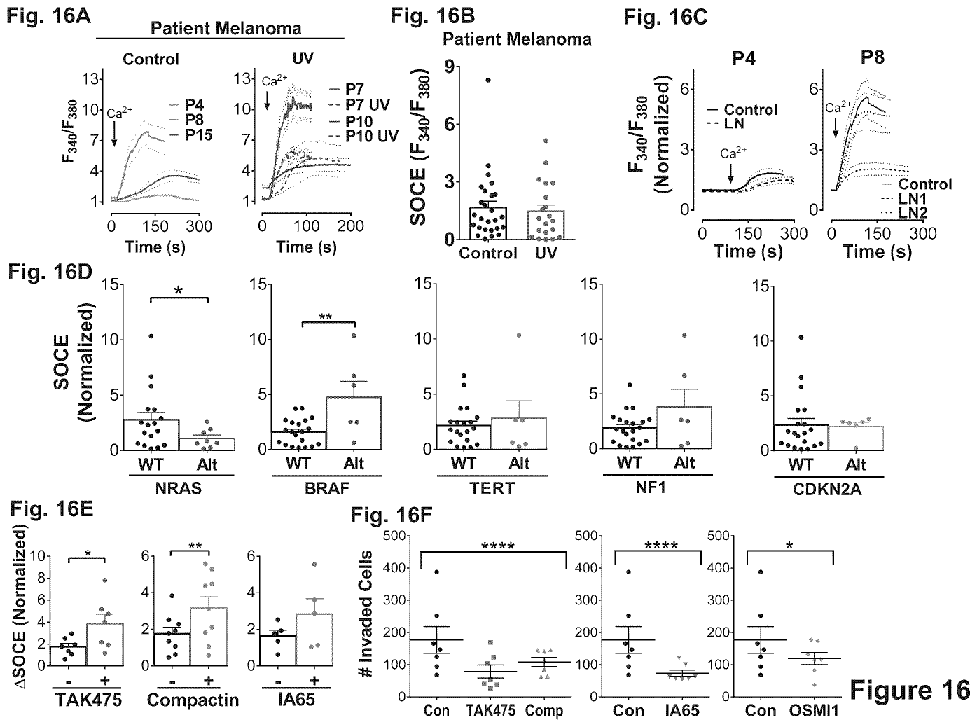

A series of freshly isolated primary patient surgical explants were used to determine the extent to which the observations reported here can be extended to human melanoma (FIG. 16). The majority of patient samples exhibited a “low SOCE” profile (FIG. 16A), similar to the SOCE suppressed samples reported elsewhere in this study. UV-irradiation failed to further suppress SOCE across all 25 samples (FIG. 16B) although the small number of samples exhibiting “high SOCE” tended to respond more to UV exposure (FIG. 16A). Both primary and metastatic lymph nodes (LN) tumor samples were obtained from two of the 25 patients (P4 and P8; FIG. 16C); the LN tumors in both patients exhibited substantially lower SOCE than the primary tumor (FIG. 16C) consistent with the concept that SOCE suppression is a component of metastasis. Finally, since all samples were genetically profiled for somatic mutations (Table 5), a link between SOCE levels and mutation status was examined. Consistent with prior studies, NRAS mutations were associated with decreased SOCE and BRAF mutations were associated with elevated SOCE (FIG. 16D) (Esteves, G. N. N., et al, 2020, Cell Calcium, 90:102241). No other relationships between SOCE and mutations were observed within the statistical power of the sample size.

TABLE 5
List of genes altered in freshly isolated patient samples.

| | Number of | Type of Alteration |
|---|---|---|
| Gene | Alterations | (Total # affected; (PT#)) |
| NRAS | 8 | Q61R (4; (3, 4, 6, 11)) |
| | | Q61L (2; (5, 27)) |
| | | Q61H (1; (13)) |
| | | Unknown mutation (1; (15)) |
| BRAF | 5 | D594N (1; (8)) |
| | | G469K (1; (10)) |
| | | V600K (1; (16)) |
| | | V600E (2; (22, 26)) |
| TERT | 6 | Expression |
| | | (6; (10, 11, 12, 13, 15, 16) |
| CDKN2A | 5 | R80* (1; (1)) |
| | | Expression (2; (11, 15) |
| | | Loss (1; (20)) |
| | | P114L (1; (30)) |
| NF1 | 5 | Q1158 (1; (8)) |
| | | Q853 (1; (8)) |
| | | R2450 (1; (10)) |
| | | Q1336 (1; (10)) |
| | | Expression (3; (11, 12, 20)) |
| FGFR2 | 1 | VUS (1; (8)) |
| FGF3/4/19 | 1 | Amplification (1; (12)) |
| ATM | 2 | N6* (1; (12)) |
| | | Unknown mutation (1; (28)) |
| CBL | 1 | E366K (1; (12)) |
| | | Alternative Splice site (1; (12)) |
| CCND1 | 1 | Amplification (1; (12)) |
| CHEK2 | 1 | S248F (1; (13)) |
| STK11 | 3 | Q302S (1; (14)) |
| | | Expression (2; (16, 20)) |
| TP53 | 1 | Expression (1; (16)) |
| LRP1B | 1 | Expression (1; (27)) |
| HRAS | 1 | Expression (1; (10)) |
| KRAS | 1 | S65N (1; (30)) |
| ROS1 | 1 | VUS (1; (8)) |
| EGFR | 1 | L862V (1; (5)) |
| PTEN | 2 | Expression (2; (3, 10)) |
| ARAF | 1 | S214F (1; (13)) |
| TET2 | 1 | Unknown mutation (1; (28)) |
| EPHA3 | 1 | Expression (1; (11)) |
| RBM10 | 1 | Expression (1; (11)) |
| EMSY | 1 | Amplification (1; (12)) |
| JAK3 | 1 | Amplification (1; (10)) |
| No specific | | 2, 7, 9, 14, 21, 23, 25, 29, 31 |
| mutations reported | | |

To assess the effect of inhibition of CHL biosynthesis and SOCE augmentation on human melanomas, several of the patient samples were treated with the mevalonate pathway inhibitors compactin and TAK475 and the Orai potentiator IA65 (FIG. 16E). Both TAK475 and compactin significantly enhanced SOCE, indicating SOCE was being suppressed by CHL biosynthesis in the clinical samples. No significant effect of IA65 was observed, although a smaller pool was tested due to limited sample availability. Finally, treatment with mevalonate pathway inhibitors compactin and TAK475, the Orai1 potentiator IA65 or the OGT inhibitor OSMI1 all led to diminished invasion in vitro (FIG. 16F). Overall, these observations reveal the clinical relevance of CHL-mediated SOCE suppression and subsequent 0-GlcNAcylation in melanoma.

### Suppression of Ca²⁺ Signaling Enhances Melanoma Progression

A single exposure to UV suppresses SOCE, which promotes melanoma migration, invasion, and metastasis. This effect is both rapid, occurring within 24 hours after UV exposure, and durable for at least 5 weeks after exposure. This effect is reversable, in that an Orai1 potentiator blocked both migration and invasion. UV-induced SOCE suppression is mediated by induction of CHL, an established inhibitor of endogenous Orai1 channel activity (Derler, I., et al., 2016, Science Signaling, 9:ra10). Finally, UV-induced SOCE suppression drives a phenotypic shift towards invasive behavior due to a combination of transcriptional and post-transcriptional changes.

Suppression of Ca²⁺ signals through UV exposure or inhibition of Orai1-STIM signaling drives invasion and metastasis; however, numerous prior investigations have observed that increased Ca²⁺ signals drive invasive behavior (Bong, A. H. L., & Monteith, G. R., 2018, 1865:1786-1794; Gross, S., et al, 2020a, Advances in Cancer Research, 148:233-317; Umemura, M., et al., 2014, PloS One, 9:e89292). While the conclusions of these investigations seem to be contradictory, it is likely a reflection of different contexts. The anti-invasive properties of Ca²⁺ are limited to a very narrow range; if SOCE is abrogated rather than attenuated, invasion does not occur. The major pathway identified in other works as responsible for pro-invasive Ca²⁺ functions is invadopodia formation (Leslie, M. 2, 2014, Journal of Cell Biology, 207:434-434; Pourfarhangi, K. E., et al, 2018, Biophysical Journal, 114:1455-1466; Sun, J., et al, 2015, Molecular & Cellular Oncology, 2:e1002714). SOCE suppression drives changes in the expression of a large number of genes associated with EMT, cell survival, contact inhibition and metabolism. Post-translational 0-GlcNAcylation drives invasive behavior in multiple cell lines and surgically collected patient explants. Given that Ca²⁺-dependent signaling is highly dependent upon concentration, context and intracellular location, this is consistent with a model in which Ca²⁺ signals can be either pro- or anti-invasive depending on cell type, degree of change and the signaling environment.

RNAseq analysis of UV- and SOCE suppression-sensitive genes revealed Aryl Hydrocarbon Signaling, Glucose Metabolism Disorder, and Impaired Glucose Metabolism as 3 of the top hits (Table 5). It is interesting to note that these pathways are associated with altered insulin sensitivity leading to increased glucose uptake (Natividad, J. M., et al, 2018, Cell Metabolism, 28:737-749.e734; Wang, C., et al, 2011, Environmental Health Perspectives, 119:1739-1744), which ultimately does occur in these cells. Within glucose metabolism disorder pathways, many genes were altered including those associated with glucose uptake, insulin sensitivity, glucose homeostasis, GLUT4 translocation and regulation of pyruvate dehydrogenase (Table 5). It was observed that 7 genes associated with EMT were upregulated, and the activity of some EMT genes (eg. Snail, c-myc, YAP, and NF-κB) is driven by O-GlcNAcylation (Akella et al., 2019, BMC Biology, 17:52). Further, a shift to HBP could drive other pro-EMT post-translational modifications such as TGFβ glycosylation (Akella, N. M., et al., 2019, BMC Biology, 17:52). Considered collectively, these observations demonstrate that SOCE suppression drives a shift to EMT through coordinated changes in both transcriptional and post-translational events.

In considering the lack of effect of UV exposure on SOCE in patient melanoma samples, it is important to recognize that the series of human cell lines exhibiting UV-induced SOCE suppression in FIG. 1A were all non-invasive. This is because it was established that invasive melanoma cells exhibit lower SOCE in a prior study (Hooper, R., et al, 2015, Molecular and Cellular Biology, 35:2790-2798)); the goal of this investigation was to examine the relationship between SOCE suppression and invasiveness within the same cell lines. Since a selection process like this was not possible with freshly isolated surgical explants, the likely explanation for the lack of effect of UV on SOCE is that the majority of the patients that donated melanoma samples for this study were afflicted with an invasive form of this disease.

The connection between UV and CHL production established here is also of clinical relevance. The introduction and wide-spread use of statins for the control of clinical CHL levels has provided an opportunity to assess the relationship between CHL and cancer progression. Statins have been shown to significantly decrease melanomagenesis (Lee, H. J., et al, 2016, Experimental Dermatology, 25:820-822) and slow progression (Pich, C., et al, 2013, Frontiers in Immunology, 4:62), although, they cannot decrease the number or clinical profile of pre-existing dyplastic nevi (Linden, K. G., et al, 2014, Cancer Prevention Research, 7:496-504). Further, it was found that melanoma metastasis could be suppressed in mice using therapeutics including LXRb (a CHL sensor/transcription factor) agonists GW3965 or T0901317 (Pencheva, N., et al., 2014, Cell, 156:986-1001; Zhao, C. & Dahlman-Wright, K., 2010, Journal of Endocrinology)), and statins (Favero, G. M., et al, 2010, Lipids in Health and Disease, 9:142; Tsubaki, M., et al, 2015, American Journal of Cancer Research, 5:3186-3197) which target CHL biosynthetic pathways. Here it is shown that UV exposure leads to upregulation of the mevalonate pathway and increases cellular CHL levels. Since blocking the mevalonate pathway blocked invasion in vitro and metastasis and high CHL in vivo was sufficient to promote metastasis independent of UV, supporting the concept that CHL serves a critical role as a mediator of UV-induced melanoma metastasis and reveals a new mechanism through which CHL promotes metastasis; SOCE inhibition. Hence, blockade of the mevalonate pathway completely reversed UV-induced SOCE inhibition and invasiveness in vitro. Since SOCE inhibition was sufficient to drive invasiveness both in vitro and in vivo, these data reveal SOCE suppression as a new mechanism for CHL-induced metastasis. CHL has previously been shown to bind directly to Orai1 where it interferes with the STIM-mediated transition to the open state (Derler, I., et al., 2016, Science Signaling, 9:ra10; Hooper, R. et al, 2016, Science Signaling, 9:fs4). Interestingly, the effect of CHL on SOCE appears to be unidirectional with CHL serving as an endogenous upstream inhibitor SOCE.

It was notable that the majority of samples tested exhibited a “low SOCE” phenotype, showing no change in SOCE in response to UV exposure. Previously, it was observed that invasive melanoma exhibits a “low SOCE” phenotype, while non-invasive cells have substantially more SOCE (Hooper, R., et al, 2015, Molecular and Cellular Biology, 35:2790-2798). In the current study, only non-invasive melanoma cell lines exhibiting “higher SOCE” were exposed to UV and tested, as the focus of the work was to determine if SOCE had a mediatory role in UV-induced melanoma progression. However, this principle was not applied to obtaining patient surgical explants; here, all samples that were made available were tested. Further, since the clinic retains tissue samples for pathological analysis and genetic profiling, patients with relatively small lesions had insufficient material; as such, only samples from relatively advanced patient samples, likely creating a selection bias towards more advanced melanoma, were obtained.

Here a previously unknown relationship between UV, CHL biosynthesis, Ca′ signaling and invasive behavior has been defined. While CHL biosynthetic pathways have been proposed to drive melanoma progression previously, the molecular context for why CHL biosynthesis might be dysregulated in melanoma had not previously been defined. Considered collectively, the relationship between Ca²⁺ signals and tumor progression is less linear than previously believed with Ca²⁺ signals serving anti-invasive role in melanoma progression. It is interesting to note that, unlike most tissues, melanocytes grow within the low extracellular Ca²⁺ concentration of the basal layer of skin. If so, the suppression of Ca²⁺ entry may be an adaptation used by melanoma cells to tolerate the high extracellular Ca²⁺ content of non-native tissues. whether the anti-invasive properties of Ca²⁺ signals are a property unique to melanoma and/or other skin tumors is not known and is the topic of ongoing investigations.

What is claimed is:

1. A method of treating a disease or disorder associated with reduced levels of Ca²⁺ signaling in need thereof, comprising administering the to the subject a composition comprising at least one modulator of store-operated Ca²⁺ entry (SOCE) in a subject in need thereof.

2. The method of claim 1, wherein the modulator of SOCE is a modulator of a member selected from the group consisting of Orai1, Orai2, and Orai3.

3. The method of claim 2, wherein the modulator of SOCE is a modulator of Orai1.

4. The method of claim 3, wherein the modulator of Orai1 is 4-((5-Phenyl-4-(trifluoromethyl)thiazol-2-yl)amino)benzoic acid (IA65) or pharmaceutically acceptable salt thereof.

5. The method of claim 1, wherein the disease or disorder associated with reduced levels of Ca²⁺ signaling is cancer.

6. The method of claim 1, wherein the disease or disorder associated with reduced levels of Ca²⁺ signaling is selected from the group consisting of basal cell carcinoma, squamous cell carcinoma, Merkel cell carcinoma, and melanoma.

7. The method of claim 6, wherein the disease or disorder is melanoma.

8. The method of claim 6, wherein administering the composition to the subject downregulates anabolic metabolic pathways in cancerous cells.

9. The method of claim 6, wherein administering the composition to the subject reduces invasiveness of the melanoma.

10. The method of claim 6, wherein administering the composition to the subject reduces migration of the melanoma.

11. The method of claim 6, wherein administering the composition to the subject reduces metastasis of the melanoma.

12. The method of claim 6, wherein the modulator of SOCE is a modulator of a member selected from the group consisting of Orai1, Orai2, and Orai3.

13. The method of claim 6, wherein the modulator of SOCE is a modulator of Orai1.

14. The method of claim 6, wherein the modulator of Orai1 is 4-((5-Phenyl-4-(trifluoromethyl)thiazol-2-yl)amino)benzoic acid (IA65) or pharmaceutically acceptable salt thereof.

15. The method of claim 14, wherein administering the composition to the subject elevates SOCE levels of cancerous cells to the levels of non-cancerous cells.

16. The method of claim 14, wherein administering the composition to the subject downregulates anabolic metabolic pathways in cancerous cells.

17. The method of claim 14, wherein administering the composition to the subject reduces the invasiveness of the melanoma.

18. The method of claim 14, wherein administering the composition to the subject reduces migration of the melanoma.

19. The method of claim 14, wherein administering the composition to the subject reduces metastasis of the melanoma.

20. The method of claim 14, wherein administering the composition to the subject does not alter SOCE levels of non-cancerous cells.
